SEQUENCE LISTING (44 sequences)

SEQ ID NO: 1 - DNA, 423 bp, Solobacterium sp.
cctatttgctccccacgctttcgtgcctcagcgtcagtctctgtctaggtagccgccttcgccaccggtgttcctccatatatctacgcattttaccgctacacatggaattccactaccctctccagtactctagtctgtcagtatccatggctttatggggttgagccccacgctttcaccatggacttagcagaccgcctacgcaccctttacgcccaataattccggataacgctcgccacctacgtattaccgcggctgctggcacgtagttagccgtggctttctggtaaggtaccgtcacgataagattctctcctatccgttctttccttacaacagagttttacaatccgaaaaccttcttcactcacgcggcgttgctcggtcagggtttcccccattgccgaaaattcccta

SEQ ID NO: 2 - DNA, 410 bp, Olsenella sp.
cctgttcgctcccctagctttcgctcctcagcgtcagtgctggcccagaaggccgccttcgccaccggtgttcttccgaatatctgcgcattccaccgctacactcggaattccgccttcccctaccacactcaagtccgccggtatcgggagcgggcgggggttgagcccccggctttgactcccgacctaacggaccgcctacgagcgctttacgcccaatgaatccggataacgctcgccccctacgtattaccgcggctgctggcacgtagttagccggggcttcttctgcaggtaccgtcacctcgcggcctcgtccctgctgaaagcggtttacgacccgaaggccttcgtcccgcacgcggcgtcgctgcgtcaggctttcgcccattgcgcaagattcccca

SEQ ID NO: 3 - DNA, 406 bp, Anaerobiospirillum succiniciproducens
cctgtttgctccccacgctttcgcacctcagcgtcagtatttgcccaggagaccgccttcgcctctggtgttctttcagatatctacgcattccaccgctacacctgaaattccatctccctctgcaatactctagaaaggcagtttagaatgaccaacccaggttgagcccgggtatttcacatcctacttacctttccgcctacatgccctttacgcccagttattccgattaacgcttgcaccctccgtattaccgcggctgctggcacggagttagccggtgcttcttctgtgggtaacgtcagtgtttcctccccactgaaagtgctttacaacccgaaggccttcttcacacacgcggcatggctgcatcagggtttcccccattgtgcaatattcccta

SEQ ID NO: 4 - DNA, 404 bp, Ruminococcus gnavus
cctgtttgctccccacgctttcgagcctcaacgtcagtcatcgtccagaaagccgccttcgccactggtgttcctcctaatatctacgcatttcaccgctacactaggaattccgctttcctctccgacactctagcctgacagttccaaatgcagtcccggggttgagccccgggctttcacatctggcttgccatgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcatcgctcacgcggcgtcgctgcatcagggtttcccccattgtgcaatattcccca

SEQ ID NO: 5 - DNA, 429 bp, Eubacterium biforme
cctatttgctccccacgctttcgtgcttcagtgtcagaatccagaccagacggccgccttcgccaccggtgttcttccatatatctacgcattttaccgctacacatggagttccgccgtcctcttctgttctctagctgatcagtttccagagcaagtacgggttgagcccataccttttactccagacttgatctgccacctacgcaccctttacgcccaatcattccggataacgctcgccacctacgtattaccgcggctgctggcacgtagttagccgtgactttctggtaagataccatcactcactcatcattccctatgagtgccgtttttctcttacaacagagctttacgatccgaagaccttcctcactcacgcggcattgctcgttcagggttccccccattgacgaaaattcccta

SEQ ID NO: 6 - DNA, 404 bp, Collinsella bouchesdurhonensis
cctgttcgctcccccagctttcgcgcctcagcgtcggtcacggcccagagggccgccttcgccaccggtgttccacccgatatctgcgcattccaccgctacaccgggtgttccaccctcccctaccggacccaagcccggaggttccgggggcgggcgggggttgagcccccgcatttgacccccggcctgccaggccgcctacgcgcgctttacgcccaatgaatccggataacgctcgccccctacgtattaccgcggctgctggcacgtagttagccggggcttcttctgcaggtacagtcttgtctcttccctgctgaaagcggtttacgacccgaaggcctccgtcccgcacgcggcgtcgctgcgtcagggttgcccccattgcgcaagattcccca

SEQ ID NO: 7 - DNA, 411 bp, Unknown
  source: /note=“Description of Unknown Bifidobacterium avesanii-like bacterium sequence”
cctgttcgctccccacgctttcgctcctcagcgtcagtaacggcccagagacctgccttcgccattggtgttcttcccgatatctacacattccaccgttacaccgggaattccagtctcccctaccgcactccagcccgcccgtacccggcgcagatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcacccgaaggcttgctcccgatcaaaagcggtttacaacccgaaggcctccgtcccgcacgcggcgtcgctgcgtcagggttccccccattgcgcaagattcccca

SEQ ID NO: 8 - DNA, 406 bp, Eubacterium sp.
cctgtttgctccccacgctttcgtacctcagtgtcagttacagtccagaaagccgccttcgccactggtgttcctcctaatatctacgcatttcaccgctacactaggaattccgctttcctctcctgcactcaagtttgccagttcgcagggcgaacaatggttgagccattgccttaaaccctgcgcttggtaaaccacctacgtaccctttacgcccaataattccggataacgcttgccccctccgtattaccgcggctgctggcacggagttagccggggcttcctccaagggtaccgtcatttgtttcttccccaaggacagagctttacgacccaaaggccttcatcgctcacgcggcgttgctgcatcaggcttgcgcccattgtgcaatattcccca

SEQ ID NO: 9 - DNA, 404 bp, Collinsella tanakaei
cctgttcgctcccccagctttcgcgcctcagcgtcggtctcggcccagagggccgccttcgccaccggtgttcctcccgatatctgcgcattccaccgctacaccgggaattccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcaggtacagtcttgtctcttccctgctgaaagcggtttacgacccgaaggcctccatcccgcacgcggcgtcgctgcgtcagggttgcccccattgcgcaagattcccca

SEQ ID NO: 10 - DNA, 404 bp, Collinsella sp.
cctgttcgctcccccagctttcgcgcctcagcgtcggtctcggcccagagggccgccttcgccaccggtgttccacccgatatctgcgcattccaccgctacaccgggtgttccaccctcccctaccggacccgagcccggcggttcagggggcgggacggggttgagccccgccatttgaccccctgcctgccgggccgcctacgcgcgctttacgcccaatgaatccggataacgctcgccccctacgtattaccgcggctgctggcacgtagttagccggggcttcttctgcaggtaccgtcttgtctcatccctgctgaaagcggtttacgacccgagggccttcgtcccgcacgcggcgtcgctgcgtcagggttccccccattgcgcaagattcccca

SEQ ID NO: 11 - DNA, 404 bp, Blautia obeum
cctgtttgctccccacgctttcgagcctcaacgtcagttaccgtccagtaagccgccttcgccactggtgttcctcctaatatctacgcatttcaccgctacactaggaattccgcttacccctccggcactcaagtatgacagtttccaatgcagtccacaggttgagcccatgcctttcacatcagacttgccacaccgtctacgctccctttacacccagtaaatccggataacgcttgccccctacgtattaccgcggctgctggcacgtagttagccggggcttcttagtcaggtaccgtcactatcttccctgctgatagaagtttacataccgagatacttcttccttcacgcggcgtcgctgcatcagggtttcccccattgtgcaatattcccca

SEQ ID NO: 12 - DNA, 428 bp, Eubacterium cylindroides
cctatttgctccccacgctttcgtacttcagcgtcagtttcagaccaggcaaccgccttcgccaccggtgttcttccatatatctacgcattttaccgctacacatggagttccattgccctcttctgcactctagcttgacagtttccagagcatactacagttgagctgtagcctttcactccagacttatcatgccgcctacgtaccctttacgcccaatcattccggataacgctcgccacctacgtattaccgcggctgctggcacgtagttagccgtgactttctggtgagataccatcaaatacatatcattgcctatatatatcctttttctctcacaacagagctttacaatccgaagaccgtcttcactcacgcggcattgctcgttcagggttccccccattgacgaaaattcccta

SEQ ID NO: 13 - DNA, 429 bp, Unknown
  source: /note=“Description of Unknown Dialister/Bacteroides xylanisolvens bacterium sequence”
cctgtttgctccccacgctttcgcgcctcagcgtcagttgtcgtccagaaagtcgccttcgccaccggtgttcttcctaatctctacgcatttcaccgctacactaggaattccactttcctctccgatactccagcctctcagtttccatcccatcacggggttaagccccgcacttttaagatggacttaagaagccgcctgcgcgcgctttacgcccaataattccggacaacgcttgccacctacgtattaccgcggctgctggcacgtagttagccgtggcttgctattcgggtaccgtcaattcagcagattattcgtctgccgaccgttcgtccccgatcacagagctttacaacccgaaggccgtcatcactcacgcggcgttgctccgtcaggctttcgcccattgcggaagattcccca

SEQ ID NO: 14 - DNA, 429 bp, Lactobacillus ruminis
cctgtttgctacccacgctttcgaacctcagcgtcagttacagaccagagagccgctttcgccactggtgttcttccatatatctacgcatttcaccgctacacatggagttccactctcctcttctgcactcaagtcttccagtttccaatgcactacttcggttaagccgaaggctttcacatcagacttaaaagaccgcctgcgttccctttacgcccaataaatccggacaacgctcgccacctacgtattaccgcggctgctggcacgtagttagccgtggctttctggtcagataccgtcaatacgtgaacagttactctcacgcacgttcttctctgacaacagaattttacgacccgaaggccttcttcattcacgcggcgttgctccatcagactttcgtccattgtggaagattcccta

SEQ ID NO: 15 - DNA, 404 bp, Unknown
  source: /note=“Description of Unknown Collinsella tanakaei-like bacterium sequence”
cctgttcgctcccccagctttcgcgcctcagcgtcggtctcggcccagagggccgccttcgccaccggtgttccacccgatatctgcgcattccaccgctacaccgggtgttccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcaggtaccgtcttgcctcatccctgctgaaagcggtttacgacccgagggccttcgtcccgcacgcggcgtcgctgcgtcagggttccccccattgcgcaagattcccca

SEQ ID NO: 16 - DNA, 405 bp, Adlercreutzia sp.
cctgttcgctcccccggctttcgcgcctcagcgtcagtgtcggcccagcaggctgccttcgccatcggtgttcctcccgatatctgcgcattccaccgctacaccgggaattccacctgcccctaccgcactcgagccgcccagttcggaacccggccgcggggttgagccccgggattagaggttccgcttaggcggccgcctacgcgcgctttacgcccaatgaatccggataacgctcgccccctacgtattaccgcggctgctggcacgtagttagccggggcttcttctgcaggtaccgtctcggttcttccctgctgaaagcggtttacaacccgaaggccttcgtcccgcacgcggcgttgctgcgtcagggttgcccccattgcgcaaaattcccca

SEQ ID NO: 17 - DNA, 404 bp, Subdoligranulum sp.
cctgtttgctacccatgctttcgagcctcagcgtcagttggtgcccagtaggccgccttcgccactggtgttcctcccgatatctacgcattccaccgctacaccgggaattccgcctacctctgcactactcaaggccagcagttttgaaagcaatttatgggttgagcccatagatttcacttccaacttgccggtccgcctgcgctccctttacacccagtaattccggacaacgcttgccacctacgttttaccgcggctgctggcacgtagttagccgtggctttcttgtacggtaccgtcattatcgtcccgtacgacaggagtttacaatccgaaaaccttcttcctccacgcggcgtcgctgcatcagggtttcccccattgtgcaatatccccca

SEQ ID NO: 18 - DNA, 404 bp, Clostridium perfringens
cctgtttgctccccacgctttcgagcctcagcgtcagttacagtccagagaatcgccttcgccactggtgttcttcctaatctctacgcatttcaccgctacactaggaattccattctcctctcctgcactctagatatccagtttggaatgcagctcccaagttaagctcgggtatttcacatcccacttaaatatccgcctacgctccctttacgcccagtaaatccggataacgcttgccacctacgtattaccgcggctgctggcacgtagttagccgtggcttcctccttaggtaccgtcattatcgtccctaaagacagagctttacaatccgaagaccttcatcactcacgcggcgttgctgcatcagggtttcccccattgtgcaatattcccca

SEQ ID NO: 19 - DNA, 404 bp, Collinsella tanakaei
cctgttcgctcccccagctttcgcgcctcagcgtcggtctcggcccagagggccgccttcgccaccggtgttcctcccgatatctgcgcattccaccgctacaccgggaattccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcgggtaccgtcttgactcgtccccgctgaaagcggtttacgacccgaaggccttcgtcccgcacgcggcgtcgctgcgtcagggtttcccccattgcgcaatattcccca

SEQ ID NO: 20 - DNA, 406 bp, Mobilibacterium massiliense
cctgtttgctccccacgctttcgtgcctcagtgtcagttgcagtccagaaagccgccttcgccgctggtgttcctcctaatatctacgcatttcaccgctacactaggaattccgctttcccctcctgcactcaagttatgcagttcgcaaggcgaacagtggttaagccactgccttaaacctcacgcttacataaccacctacgcactctttacgcccaataattccggataacgcttgccccctacgtattaccgcggctgctggcacgtagttagccggggcttgctgcccgggtaccgtcactttcttcgtccccgggaacagaagtttacaacccgaaggccttcatccttcacgcggcgttgctgcatcaggctttcgcccattgtgcaagattcccta

SEQ ID NO: 21 - DNA, 404 bp, Unknown
  source: /note=“Description of Unknown Sellimonas/Drancourtella/Ruminococcus/Lachnospiraceae bacterium sequence”
cctgtttgctccccacgctttcgagcctcaacgtcagtcaccgtccagtaagccgccttcgccactggtgttcctcctaatatctacgcatttcaccgctacactaggaattccgcttacctctccggcactccagaattgcagtttccaaagcagtcccagggttgagccctgggttttcactccagacttgcctctccgtctacgctccctttacacccagtaaatccggataacgcttgcaccatacgtattaccgcggctgctggcacgtatttagccggtgcttcttagtcaggtaccgtcagtttcttccctgctgatagaagtttacataccgaaatacttcttccttcacgcggcgtcgctgcatcagggtttcccccattgtgcaatattcccca

SEQ ID NO: 22 - DNA, 429 bp, Streptococcus lutetiensis
cctgtttgctccccacgctttcgagcctcagcgtcagttacagaccagagagccgctttcgccaccggtgttcctccatatatctacgcatttcaccgctacacatggaattccactctccccttctgcactcaagtctaacagtttccaaagcgaacaatggttaagccactgcctttaacttcagacttattaaaccgcctgcgctcgctttacgcccaataaatccggacaacgctcgggacctacgtattaccgcggctgctggcacgtagttagccgtccctttctggtaagttaccgtcactgtgtgaactttccactctcacacacgttcttctcttacaacagagctttacgatccgaaaaccttcttcactcacgcggcgttgctcggtcagggttgcccccattgccgaagattcccta

SEQ ID NO: 23 - DNA, 404 bp, Unknown
  source: /note=“Description of Unknown Lachnospiraceae NK4A136 family bacterium sequence”
cctgtttgctccccacgctttcgagcctcaacgtcagttaccgtccagtaagccgccttcgccactggtgttcttcctaatatctacgcatttcaccgctacactaggaattccgcttacctctccggcactccagctaaacagtttccaaagcagtcccggcgttaagcaccgggctttcacttcagacttgccttgccgtctacactccctttacacccagtaaatccggataacgcttgccccctacgtattaccgcggctgctggcacgtagttagccggggcttcttattcaggtaccgtcactttcttccctgctgatagagctttacataccgaaatacttcttcactcacgcggcgtcgctgcatcagggtttcccccattgtgcaatattcccca

SEQ ID NO: 24 - DNA, 404 bp, Unknown
  source: /note=“Description of Unknown Novel Lachnospiraceae family bacterium sequence”
cctgtttgctccccacgctttcgagcctcaacgtcagttaccgtccagtaagccgccttcgccactggtgttcctcctaatatctacgcatttcaccgctacactaggaattccgcttacctctccggcactccagcagaacagtttccaatgcaggtccggggttgagccccggaatttcacatcagacttgctccgccgtctacgctccctttacacccagtaaatccggataacgcttgccccctacgtattaccgcggctgctggcacgtagttagccggggcttcttagtcaggtaccgtcattatcttccctgctgatagagctttacataccgaaatacttcttcgctcacgcggcgtcgctgcatcagggtttcccccattgtgcaatattcccca

SEQ ID NO: 25 - DNA, 404 bp, Eubacterium sp.
cctgtttgctccccacgctttcgtgcctcagcgtcagttccagtccagcaggccgccttcgccactggtgttcctcctaatatctacgcatttcaccgctacactaggaattccgcctgcctctcctggactccagcaaaacagtttcaaatgcagccccggagttgagccccggtctttcacatctgacttgccttgccgcctacgcaccctttacacccagtaaatccggataacgcttgctccatacgtattaccgcggctgctggcacgtatttagccggagcttcttagtcaggtaccgtcattatcttccctgctgatagagctttacataccgaaatacttcttcactcacgcggcgttgctgcatcagggtttcccccattgtgcaatattcccca

SEQ ID NO: 26 - DNA, 406 bp, Unknown
  source: /note=“Description of Unknown Subdoligranulum variabile/Clostridiales bacterium 40-4c/ Gemmiger formicilis bacterium sequence”
cctgtttgctacccatgctttcgagcctcagcgtcagttggtgcccagtaggtcgccttcgccactggtgttcctcccgatatctacgcattccaccgctacaccgggaattccacctacctctgcactactcaaggccagcagttttgaaagcaatttatgggttgagcccatagatttcactcccaacttgccggtccgcctgcgctccctttacacccagtaattccggacaacgcttgcaccctacgttttaccgcggctgctggcacgtagttagccggtgctttcttgttaggtaccgtcagatattcgtccctaacgacaggagtttacaatccgaaaaccttcttcctccacgcggcgtcgctgcatcagggtttcccccattgtgcaatatccccca

SEQ ID NO: 27 - DNA, 429 bp, Dialister succinatiphilus
cctgtttgctccccacgctttcgcgcctcagcgtcagttgtcgtccagaaagtcgccttcgccaccggtgttcttcctaatctctacgcatttcaccgctacactaggaattccactttcctctccgatactccagcttcccagtttccatcccatcacggggttaagccccgcacttttaagatggacttagaaagccgcctgcgcgcgctttacgcccaataattccggacaacgcttgccacctacgtattaccgcggctgctggcacgtagttagccgtggcttgctattcgggtaccgtcacttccataaggtattagcttacggaccgttcgtccccgatcacagagctttacaacccgaaggccgtcatcactcacgcggcgttgctccgtcaggctttcgcccattgcggaagattcccca

SEQ ID NO: 28 - DNA, 423 bp, Solobacterium sp.
cctatttgctccccacgctttcgtgcctcagcgtcagtctctgtctaggtagccgccttcgccaccggtgttcctccatatatctacgcattttaccgctacacatggaattccactaccctctccagtactctagcctgccagtatctatggctttatggggttgagccccacgctttcaccataaacttaacaggccgcctacgcaccctttacgcccaataattccggataacgctcgctacctacgtattaccgcggctgctggcacgtagttagccgtagctttctggtaaggtaccgtcactcacctattctctagatgccgttctccccttacaacagagctttacaatccgaagaccttcatcactcacgcggcgttgctcggtcagggttgcccccattgccgaaaattcccta

SEQ ID NO: 29 - DNA, 404 bp, Clostridium sp.
cctgtttgctccccacgctttcgagcctcaacgtcagttatcgtccagcaagccgccttcgccactggtgttcctcctaatatctacgcatttcaccgctacactaggaattccgcttgcctctccgacactccagctgcacagtttccaaagcagtcccggggttgagccccgggctttcacttcagacttgcactgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 30 - DNA, 405 bp, Eubacterium sp.
cctgtttgctccccacactttcgagcctcagcgtcagttaaagcccagtaggccgccttcgccaccggtgttcctccgaatatctacgcatttcaccgctacactcggaattccgcctacctctacttcactcaagaaaaccagtttcaactgcagtctacaagttaagcccgtagttttcacagctgacttggcttcccgcctgcgctccctttacacccagtaattccggacaacgcttgccacctacgtattaccgcggctgctggcacgtagttagccgtggcttgctccttagctaccgtcaaaattcttcactaagaacagaagtttacaatccgaaaaccgtcttccttcacgcggcgttgctgcatcagggtttcccccattgtgcaatattcccca

SEQ ID NO: 31 - DNA, 429 bp, Acidaminococcus sp.
cccgtttgctaccctggctttcgcatctcagcgtcagacacagtccagaaaggcgccttcgccactggtgttcctcccaatatctacgcatttcaccgctacactgggaattcccctttcctctcctgcactcaagacttccagtatccaacgccatacggggttaagccccgcattttcacgtcggacttaaaagcccgcctacatgctctttacgcccaataattccggacaacgcttgccacctacgtattaccgcggctgctggcacgtagttagccgtggcttcctcgtcaggtaccgtcaacaccaaaatgtattatagatcggtgcattcgtccctgacaacagagttttacaatccgaagaccttcatcactcacgcggcgttgctccgtcagactttcgtccattgcggaagattcccca

SEQ ID NO: 32 - DNA, 429 bp, Megasphaera sp.
cccgtttgctcccctggctttcgcgcctcagcgtcagttgtcgtccagaaagccgctttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcacttacgacgggtattgaccgtcgtaccattcgtcccgtataacagaactttacaacccgaaggccgtcatcgttcacgcggcgttgctccgtcagactttcgtccattgcggaagattcccca

SEQ ID NO: 33 - DNA, 429 bp, Megasphaera sp.
cccgtttgctcccctggctttcgcgcctcagcgtcagttgtcgtccagaaagccgctttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcacttacgacgggtattgaccgtcgtaccattcgtcccgtataacagaactttacaacccgaaggccgtcatcccgcacgcggcgtcgctgcatcaggcttgcgcccattgtgcaatattcccca

SEQ ID NO: 34 - DNA, 429 bp, Megasphaera sp.
cctgtttgctccccacgctttcgagcctcaacgtcagttaccgtccagtaagccgccttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcacttacgacgggtattgaccgtcgtaccattcgtcccgtataacagaactttacaacccgaaggccgtcatcgttcacgcggcgttgctccgtcagactttcgtccattgcggaagattcccca

SEQ ID NO: 35 - DNA, 409 bp, Bifidobacterium sp.
cctgttcgctccccacgctttcgctcctcagcgtcagtaacggcccagagacctgccttcgccattggtgttcttcccgatatctacacattccaccgttacaccgggaattccagtctcccctgccgcactccagcccgcccgtacccggcgcagatccaccgttaagcgatggactttcacaccagacgcgacgaaccgcctacgagccctttacgcccaataaatccggataacgcttgcaccctacgtattaccgcggctgctggcacgtagttagccggtgcttattcaacgggtacactcactctcgcttgctccccgataaaagcggtttacaacccgaaggcctccatcccgcacgcggcgtcgctgcgtcaggctttcgcccattgcgcaatattcccca

SEQ ID NO: 36 - DNA, 410 bp, Unknown
  source: /note=“Description of Unknown Coriobacteriaceae novel family bacterium sequence”
cctgttcgctcccccagctttcgcgcctcagcgtcagtcgtggcccagagggccgccttcgccaccggtgttcttcccaatatctgcgcattccaccgctacactgggaattccaccctcccctgccagactcaagccccgtggttccgggagcgggcgggggttgagcccccggatttgactcccggcctacgaggccgcctacgcgcgctttacgcccaatgaatccggataacgctagccccatacgtattaccgcggctgctggcacgtatttagccggggcttcttctgcaggtaccgtcaccttgcggcctcttccctgctgaaagcggtttacgacccgagggccttcgtcccgcacgcggcgtcgctgcgtcagggttccccccattgcgcaagattcccca

SEQ ID NO: 37 - DNA, 411 bp, Unknown
  source: /note=“Description of Unknown Bifidobacterium adolescentis-like bacterium sequence”
cctgttcgctccccacgctttcgctcctcagcgtcagtaacggcccagagacctgccttcgccattggtgttcttcccgatatctacacattccaccgttacaccgggaattccagtctcccctaccgcactccagcccgcccgtacccggcgcagatccaccgttaagcgatggactttcacaccggacgcgacgaaccgcctacgagccctttacgcccaataattccggataacgcttgcaccctacgtattaccgcggctgctggcacgtagttagccggtgcttattcgaaaggtacactcacccgaaggcttgctcccgatcaaaagcggtttacaacccgaaggccgtcatcccgcacgcggcgtcgctgcatcaggcttgcgcccattgtgcaatattcccca

SEQ ID NO: 38 - DNA, 428 bp, Unknown
  source: /note=“Description of Unknown Catenibacterium mitsuokai like bacterium sequence”
cctatttgctccccacgctttcgggaatgagcgtcagttgcggcccagaccgtcgccttcgccactggtgttcctccatatatctacgcatttcaccgctacacatggaattccacgatcctcttccgcactctagccgcccggtttccatggcttaccgaagtttagcttcggtctttcaccacagacccttgcggccgcctgctccctctttacgcccaatgattccggataacgctcgccacctacgtattaccgcggctgctggcacgtagttagccgtggctttctggtaaagtaccgtcactcgcataccatttcctgtatgcgccgttcttcctttacaacagagctttacgacccgaaggccttcttcgctcacgcggcgttgctcggtcagggtttcccccattgccgaaaattcccta

SEQ ID NO: 39 - DNA, 404 bp, Unknown
  source: /note=“Description of Unknown Candidatus Dorea massiliensis-like bacterium sequence”
cctgtttgctccccacgctttcgagcctcaacgtcagttactgtccagtaagccgccttcgccactggtgttcctcctaatatctacgcatttcaccgctacactaggaattccacttacctctccagcactctagcctgacagtttccaaagcagtcccggggttgagccccgggctttcacttcagacttgccatgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcaggctttcgcccattgtgcaatattcccca

SEQ ID NO: 40 - DNA, 404 bp, Blautia producta
cctgtttgctccccacgctttcgagcctcaacgtcagttaccgtccagtaagccgccttcgccactggtgttcctcctaatatctacgcatttcaccgctacactaggaattccgcttacctctccggtactcaagatcaacagtttccaatgcagtccgggggttgagcccccgcctttcacatcagacttgctgctccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 41 - DNA, 408 bp, Unknown
  source: /note=“Description of Unknown Coriobacteriaceae family UCG-002 bacterium sequence”
cctgttcgctcccccagctttcgctcctcagcgtcagtgatggcccagaaagccgccttcgccaccggtgttcttcccgatatctgcgcattccaccgctacaccgggaattccgctttcccctaccacactcaagtccaccggtatcgggggcggaccggggttgagccccgggatttgacctccgacctaatggaccgcctacgagcgctttacgcccaatgaatccggataacgctcgccccctacgtattaccgcggctgctggcacgtagttagccggggcttcttctgcaggtaccgtcactttcgcctcgtccctgctgaaagcggtttacgacccgaaggccttcatcccgcacgcggcgtcgctgcgtcaggctttcgcccattgcgcaagattcccca

SEQ ID NO: 42 - DNA, 404 bp, Anaerostipes sp.
cctgtttgctccccacgctttcgtgcctcagtgtcagtttcagtccagtaagccgccttcgccactgatgttcctcctaatatctacgcatttcaccgctacactaggaattccgcttacctctcctgcactccagttcgacagtttcaaaagcagtcccgcggttaagccccgggctttcacttctgacttgccgtaccacctacgcaccctttacacccagtaattccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagggttccccccattgtgcaatattcccca

SEQ ID NO: 43 - DNA, 58 bp, Artificial Sequence
  source: /note=“Description of Artificial Sequence Synthetic primer”
caagcagaagacggcatacgagatgtgactggagttcagacgtgtgctcttccgatct

SEQ ID NO: 44 - DNA, 58 bp, Artificial Sequence
  source: /note=“Description of Artificial Sequence Synthetic primer”
aatgatacggcgaccaccgagatctacactctttccctacacgacgctcttccgatct

(19) United States
(12) Patent Application Publication
(10) Pub. No.: US 2022/0073970 A1
(43) Pub. Date: Mar. 10, 2022

(54) MONITORING AND DIAGNOSTIC METHODS FOR FELINE MICROBIOME CHANGES

(71) Applicant: MARS, INCORPORATED, McLean, VA (US)

(72) Inventors: Zoe V. MARSHALL-JONES, Melton Mowbray, Leicestershire (GB); Ruth STAUNTON, Melton Mowbray, Leicestershire (GB); Richard HAYDOCK, Melton Mowbray, Leicestershire (GB); Ciaran O'FLYNN, Melton Mowbray, Leicestershire (GB); Phillip WATSON, Melton Mowbray, Leicestershire (GB)

(73) Assignee: MARS, INCORPORATED, McLean, VA (US)

(21) Appl. No.: 17/423,663

(22) PCT Filed: Jan. 20, 2020
(86) PCT No.: PCT/US2020/014307

§ 371 Date: Jul. 16, 2021

(30) Foreign Application Priority Data
Jan. 18, 2019 (GB) 1900754.1

(51) Int. Cl. C12Q 1/689

(52) U.S. Cl. CPC C12Q 1/689

(57) ABSTRACT

Methods for assessing the microbiome of a feline are provided. The methods include, inter alia, detecting one or more bacterial species in a sample obtained from the feline, comparing the one or more bacterial species to a control data set, and determining the microbiome age.

### CROSS-REFERENCE TO RELATED APPLICATIONS

This application claims priority to UK Patent Application No. 1900754.1, filed on Jan. 18, 2019, the contents of which are incorporated herein by reference in its entirety.

### TECHNICAL FIELD

The present disclosure relates to the field of monitoring tools and diagnostic methods for determining the age status of a feline's microbiome and therein assessing the health of the animal.

### BACKGROUND TO THE INVENTION

The understanding of the microbiome and its impact on health has increased significantly in recent years. Changes in the microbiome, and its interaction with the immune, endocrine and nervous systems are correlated with a wide array of health conditions and illnesses, ranging from inflammatory bowel disease [1-3] to cancer [4] and to behavioral aspects of host health [5;6].

The establishment of the microbiome occurs at the same time as development of the immune system and plays a role in intestinal physiology and regulation. The initial establishment of the gut microbiota is an essential step in neonatal development, influencing immunological development in infancy and health throughout life. As such in humans and many mammals a rapid increase in diversity occurs in the early establishment phase of gut microbiome development [7].

The adult gut microbiome can be resilient to large shifts in community structure and in humans and other mammals it is considered to be relatively stable throughout adult life. This “adult microbiome” is considered to represent a healthy gut microbiome for cats with enhanced resilience compared to other life stages. In early life stages, kittens have an undeveloped gut barrier, which includes the gastrointestinal microbiome as well as histological and gut associated immune functions. Kittens and young cats are therefore are more prone to gastrointestinal illnesses such as diarrhoea and sickness, etc. Senior and geriatric cats are also more prone to diarrhoea and gastrointestinal complications, which can occur in part as a result of a deterioration in the gut microbiome.

Given the importance of the microbiome to health and wellbeing, it is important to find ways to determine the status of the microbiome of an animal because of the inherent changes in the gut barrier and gastrointestinal health with age, this status can be considered in terms of the age status of the microbiome.

### SUMMARY OF THE INVENTION

The present disclosure features diagnostic methods which allow the determination of a feline's microbiome age status. The methods disclosed herein can achieve this with high accuracy, as shown in the examples.

In one aspect, the disclosure provides a method of determining the microbiome age status of a feline, comprising (a) detecting one or more bacterial species in a sample obtained from the feline; (b) comparing the one or more bacterial species in the sample to a control data set; and (c) determining the microbiome age status. These methods are particularly useful for assessing a feline's health as a discrepancy between the microbiome age status and the feline's actual age can be indicative of its health status. For example, it would be undesirable for an adult cat to have a juvenile microbiome as this is associated with low diversity and is associated with health conditions such as the susceptibility to diarrhoea.

In another aspect, further provided is a method of monitoring a feline, comprising a step of determining the microbiome age status of the feline by a method of the present disclosure on at least two time points. This is particularly useful where a feline is receiving treatment to shift the microbiome as it can monitor the progress of the therapy. It is also useful for monitoring health of the feline as a rapid shift from, for example, an adult microbiome to a senior microbiome, can be indicative of disease. This aspect of the method of the present disclosure can also be used to assess whether the feline's microbiome progresses as the animal gets older.

In some embodiments, the methods of the present disclosure comprise a further step of changing the composition of the microbiome. This can be achieved through a dietary change or through consumption of a functional food or administration of a nutraceutical or pharmaceutical composition or of a preparation comprising bacteria. This is particularly useful where the methods of the present disclosure have identified a microbiome age status that is not consistent with the feline's actual age and where the feline can therefore be less healthy in the context of the animal's actual age.

In another aspect, disclosed herein is a method of monitoring the microbiome age status in a feline who has undergone a dietary change and/or who has received a supplement, a functional food a nutraceutical composition or a pharmaceutical composition that is able to change the microbiome composition, comprising determining the microbiome age status by a method according to the present disclosure. Such methods allow a skilled person to determine the success of the treatment. In specific embodiments, these methods comprise determining the microbiome age status before and after treatment as this helps to evaluate the success of the treatment.

In another aspect, also provided is a method of assessing the microbiome age status of a feline to determine whether an intervention is required, comprising (a) quantitating two or more bacterial species in a sample obtained from the feline; (b) determining the relative abundance of said bacterial species; (c) comparing the relative abundance determined in step (b) to that of a control data set; wherein if the comparing of step (c) indicates a difference in microbiome age status to actual age of the feline, an intervention is recommended.

In a further aspect, also provided is a method of assessing the microbiome age status of a feline to determine whether an intervention is required, comprising (a) quantitating two or more bacterial species in a sample obtained from the feline; (b) calculating the diversity index; (c) comparing the diversity index to the diversity index of a control data set; wherein if the diversity index calculated in step (b) is less than the 5th percentile or greater than the 95th percentile of the diversity index in control data set, an intervention is recommended.

In another aspect, also provided is a method of assessing the microbiome age status of a feline to determine whether an intervention is required, comprising (a) quantitating two or more bacterial species in a sample obtained from the feline; (b) determining the relative abundance of said bacterial species; (c) calculating the Shannon index diversity by the formula:

$$Shannon Index ( H ) = - {\sum }_{i = 1}^{s} {p}_{i} ln {p}_{i}$$

wherein if the diversity calculated in step (c) is less than 2.77 or greater than 3.99 in an adult feline, less than 2.41 or greater than 3.92 in a senior feline, or less than 1.65 or greater than 4.17 in a geriatric feline, an intervention is recommended.

As noted above, the presently disclosed matter provides a method of determining the microbiome age status of a feline, comprising (a) detecting one or more bacterial species in a sample obtained from the feline; (b) comparing the one or more bacterial species in the sample to a control data set; and (c) determining the microbiome age status. In certain embodiments of the claimed method, the control data set comprises microbiome data of felines at different times or life stages. In certain embodiments of the claimed method, the control data comprises microbiome data from at least two, at least three, or four life stages of a feline selected from the list consisting of a kitten, a youth feline, an adult feline, a senior feline and a geriatric feline.

In certain embodiments of the claimed methods, the kitten is defined as up to about 1.0 years old, youth is defined as greater than about 1.0 and up to about 4.5 years old, adult feline is defined as greater than about 4.5 and up to about 9.5 years old, senior feline is defined as greater than about 9.5 and up to about 13.5 years old, and geriatric is defined as greater than about 13.5 years old.

In certain embodiments of the claimed methods, the method comprises quantitating the one or more bacterial species. In particular embodiments of the claimed methods, the bacterial species is selected from the group consisting of: Solobacterium, Olsenella, Anaerobiospirillum, Blautia, Holdemanella, Collinsella, Bifidobacterium, Eubacterium, Ruminococcus, Dialister, Bacteroides, Lactobacillus, Adlercreutzia, Subdoligranulum, Clostridium, Mogibacterium, Sellimonas, Streptococcus, Subdoligranulum and Lachnoclostridium, or bacterial species from the family Lachnospiraceae. In particular embodiments of the claimed methods, the bacterial species are selected from the group consisting of Solobacterium sp., Olsenella sp. Marseille P2300, Anaerobiospirillum succiniciproducens, Blautia gnavus, Holdemanella biforme, Collinsella bouchesdurhonensis, Bifidobacterium sp., Eubacterium brachy, Collinsella tanakaei, Collinsella stercoris, Blautia obeum, Eubacterium cylindroides, Dialister/Bacteroides xylanisolvens, Lactobacillus ruminis, Adlercreutzia sp., Subdoligranulum sp., Clostridium perfringens, Mogibacterium massiliense, Sellimonas sp., Streptococcus luteciae, Lachnospiraceae sp., Subdoligranulum variabile, Dialister succinatiphilus, Lachnoclostridium [Clostridium] leptum, Lachnoclostridium [Clostridium] hylemonae and Eubacterium coprostanoligenes. In particular embodiments of the claimed methods, the bacterial species has a 16S rDNA with at least about 95%, at least about 96%, at least about 97%, at least about 98%, at least about 99% or 100% identity to the sequence of any one of SEQ ID NOs: 1-30.

In certain embodiments of the claimed methods, the method comprises the detection of bacterial species from one or more genera selected from the group consisting of Solobacterium, Olsenella and Anaerobiospirillum. In certain embodiments of the claimed methods, the method comprises the detection of one or more bacterial species selected from the group consisting of Solobacterium sp., Olsenella sp., Marseille-P2300, and Anaerobiospirillum succiniciproducens. In particular embodiments of the claimed methods, the bacterial species have a 16S rDNA with at least about 95%, at least about 96%, at least about 97%, at least about 98%, at least about 99% or 100% identity to the sequence of any one of SEQ ID NOs: 1-3.

In certain embodiments of the claimed methods, the method further comprises the detection of one or more bacterial species selected from the group consisting Blautia gnavus, Holdemanella biforme, Collinsella bouchesdurhonensis, and Bifidobacterium avesanii. In certain embodiments of the claimed methods, the bacterial species have a 16S rDNA with at least about 95%, at least about 96%, at least about 97%, at least about 98%, at least about 99% or 100% identity to the sequence of any one of SEQ ID NOs: 4-7. In certain embodiments of the claimed methods, the method comprises the detection of all of SEQ ID NOs: 8-17. In certain embodiments of the claimed methods, the method comprises detection of a bacterial species from the genus Solobacterium. In particular embodiments of the claimed methods, the bacterium has a 16S rDNA with at least about 95%, at least about 96%, at least about 97%, at least about 98%, at least about 99% or 100% identity with the sequence of SEQ ID NO: 1.

In certain embodiments of the claimed methods, at least 5, at least 10, at least 15, at least 20, at least 25 or at least 30 different species are detected and/or quantitated.

In certain embodiments of the claimed methods, the method comprises detecting or quantitating Solobacterium sp., Olsenella sp. Marseille-P2300 and Anaerobiospirillum succiniciproducens. In certain embodiments of the claimed methods, the bacterial species have a 16S rDNA with at least about 95%, at least about 96%, at least about 97%, at least about 98%, at least about 99% or 100% identity to the sequence of any one of SEQ ID NOs: 1, 2, and 3.

In certain embodiments of the claimed methods, the method further comprises detecting or quantitating Blautia gnavus, Holdemanella biforme, Collinsella bouchesdurhonensis, and Bifidobacterium avesanii. In certain embodiments of the claimed methods, the bacterial species have a 16S rDNA with at least about 95%, at least about 96%, at least about 97%, at least about 98%, at least about 99% or 100% identity to the sequence of any one of SEQ ID NOs: 4, 5, 6 and 7.

In certain embodiments of the claimed methods, the method further comprises detecting or quantitating Eubacterium sp., Collinsella sp. Blautia sp., Dialister/Bacteroides sp., Lactobacillus sp., Adlercreutzia sp., Subdoligranulum sp.; particularly detecting or quantitating Eubacterium brachy, Collinsella tanakaei, Collinsella stercoris, Blautia obeum, Eubacterium cylindroides, Dialister/Bacteroides xylanisolvens, Lactobacillus ruminis, Adlercreutzia sp. and Subdoligranulum sp. In certain embodiments of the claimed methods, the bacterial species have a 16S rDNA with at least about 95%, at least about 96%, at least about 97%, at least about 98%, at least about 99% or 100% identity to the sequence of any one of SEQ ID NOs: 9-18.

In certain embodiments of the claimed methods, step (b) comprises correlating the one or more bacterial species in the sample to a control data set using partial least squares discriminate analysis.

In certain embodiments of the claimed methods, the bacterial species are from genera selected from the group consisting of Solobacterium, Eubacterium, Blautia, Lachnoclostridium, Olsenella, Acidaminococcus, Anaerobiospirillum, Megasphaera, Bifidobacterium, Coriobacteriaceae, Catenibacterium, Drancourtella, Sellimonas, Ruminococcus, Dorea, Collinsella, Holdemanella, Mogibacterium and Anaerostipes. In particular embodiments of the claimed methods, the bacterial species are selected from the group consisting of Solobacterium sp., Eubacterium brachy, Blautia obeum, Lachnoclostridium [Clostridium] leptum, Lachnoclostridium [Clostridium] hylemonae, Olsenella sp., Acidaminococcus fermentans, Acidaminococcus timonensis, Anaerobiospirillum succiniciproducens, Megasphaera indica, Megasphaera elsdenii, Megasphaera sp. Bifidobacterium gallinarum, Bifidobacterium pullorum, Bifidobacterium saeculare, Bifidobacterium subtile, Coriobacteriaceae sp., Bifidobacterium sp., Catenibacterium mitsuokai, Drancourtella massiliensis, Sellimonas intestinalis, Ruminococcus sp, Lachnospiraceae sp., Dorea sp., Blautia producta, Coriobacteriaceae sp, Collinsella sp., Holdemanella biforme, Mogibacterium massiliense, Anaerostipes hadrus, Anaerostipes sp., Blautia gnavus, Collinsella stercoris/intestinalis, Bifidobacterium sp. and Collinsella tanakaei. In certain embodiments of the claimed methods, the bacterial species has a 16S rDNA with at least about 95%, at least about 96%, at least about 97%, at least about 98%, at least about 99% or 100% identity to the sequence of any one of SEQ ID NOs: 1, 8, 11, 29, 2, 31, 3, 32, 33, 34, 35, 36, 37, 38, 21, 28, 39, 40, 41, 9, 5, 20, 42, 4, 10, 7 and 15. In certain embodiments of the claimed methods, the method comprises detection of a bacterial species from the genus Solobacterium. In certain embodiments of the claimed methods, the bacterium has a 16S rDNA with at least about 95%, at least about 96%, at least about 97%, at least about 98%, at least about 99% or 100% identity with the sequence of SEQ ID NO: 1.

In certain embodiments of the claimed methods, at least 5, at least 10, at least 15, at least 20, at least 25 or at least 27 different taxa are detected and/or quantitated.

In certain embodiments of the claimed methods, the method comprises detecting or quantitating Solobacterium sp., Eubacterium brachy and Blautia obeum. In certain embodiments of the claimed methods, the bacterial species have a 16S rDNA with at least about 95%, at least about 96%, at least about 97%, at least about 98%, at least about 99% or 100% identity to the sequence of any one of SEQ ID NOs: 1, 8, and 11. In certain embodiments of the claimed methods, the method further comprises detecting or quantitating Lachnoclostridium [Clostridium] leptum/Lachnoclostridium [Clostridium] hylemonae, Olsenella sp., Acidaminococcus fermentans/Acidaminococcus timonensis, and Anaerobiospirillum succiniciproducen. In certain embodiments of the claimed methods, the bacterial species have a 16S rDNA with at least about 95%, at least about 96%, at least about 97%, at least about 98%, at least about 99% or 100% identity to the sequence of any one of SEQ ID NOs: 29, 2, 31, 3.

In certain embodiments of the claimed methods, the method further comprises detecting or quantitating Megasphaera sp., Bifidobacterium sp., Coriobacteriaceae sp., Catenibacterium sp., Drancourtella sp., Sellimonas sp., Ruminococcus sp., Dorea sp. Collinsella, Holdemanella, Mogibacterium and Anaerostipes; preferably detecting or quantitating Megasphaera indica/Megasphaera elsdenii, Megasphaera sp., Bifidobacterium gallinarum/Bifidobacterium pullorum/Bifidobacterium saeculare/Bifidobacterium subtile, Coriobacteriaceae sp., Bifidobacterium sp., Catenibacterium mitsuokai, Drancourtella massiliensis/Sellimonas intestinalis/Ruminococcus sp/Lachnospiraceae sp., Dorea sp., Blautia producta, Coriobacteriaceae sp, Collinsella sp., Holdemanella biforme, Mogibacterium massiliense, Anaerostipes hadrus, Anaerostipes sp., Blautia gnavus, Collinsella stercoris/intestinalis, Bifidobacterium sp. and Collinsella tanakaei.

In an alternative embodiment, the presently disclosed subject matter provides a method, wherein the feline's microbiome age status is compared to its biological age and the feline's owner is notified when there is a discrepancy.

In an alternative embodiment, the presently disclosed subject matter provides a method of monitoring a feline, comprising a step of determining the microbiome age status of the animal by the method of any preceding claim on at least two time points. In certain embodiments of the claimed method, the two time points are at least about 6 months apart.

In certain embodiments of the claimed methods, the methods further comprise changing the composition of the microbiome by administration of a dietary change, a supplement, a functional food, a nutraceutical or a pharmaceutical composition.

In an alternative embodiment, the presently disclosed subject matter provides a method of monitoring the microbiome age status in a feline that has undergone a dietary change, and/or who has received a supplement, a functional food, a nutraceutical composition or a pharmaceutical composition, which is able to change the microbiome composition, comprising determining the microbiome age status by the method of any preceding claim. In certain embodiments of the claimed methods, the microbiome age status is determined before and after administration of the composition. In certain embodiments of the claimed methods, the composition comprises one or more bacteria.

In an alternative embodiment, the presently disclosed subject matter provides a method of assessing the microbiome age status of a feline to determine whether an intervention is required, comprising (a) quantitating two or more bacterial species in a sample obtained from the feline; (b) determining the relative abundance of said bacterial species; (c) comparing the relative abundance determined in step (b) to that of a control data set; wherein if the comparing of step (c) indicates a difference in microbiome age status to actual age of the feline, an intervention is recommended.

In an alternative embodiment, the presently disclosed subject matter provides a method of assessing the microbiome age status of a feline to determine whether an intervention is required, comprising (a) quantitating two or more bacterial species in a sample obtained from the feline; (b) calculating the diversity index; (c) comparing the diversity index to the diversity index of a control data set; wherein if the diversity index calculated in step (b) is less than the 5th percentile or greater than the 95th percentile of the diversity index in the control data set, an intervention is recommended.

In certain embodiments of the claimed methods, the control data set comprises bacterial species diversity data from a plurality of other felines of the same life stage as the feline; or bacterial species diversity data from the same feline at one or more different time points, preferably separated by at least about 6 months.

In certain embodiments of the claimed methods, the feline is an adult, senior or geriatric feline.

In an alternative embodiment, the presently disclosed subject matter provides a method of assessing the microbiome age status of a feline to determine whether an intervention is required, comprising (a) quantitating two or more bacterial species in a sample obtained from the feline; (b) determining the relative abundance of said bacterial species; (c) calculating the Shannon index diversity by the disclosed formula; wherein if the diversity calculated in step (c) is less than about 2.77 or greater than about 3.99 in an adult feline, less than about 2.41 or greater than about 3.92 in a senior feline, or less than about 1.65 or greater than about 4.17 in a geriatric feline, an intervention is recommended. In certain embodiments of the claimed methods, the adult feline is defined as greater than about 4.5 and up to about 9.5 years old, senior feline is defined as greater than about 9.5 and up to about 13.5 years old, and geriatric is defined as greater than about 13.5 years old.

In certain embodiments of the claimed methods, the intervention is a dietary change, a supplement, a functional food, a nutraceutical or a pharmaceutical composition. In certain embodiments of the claimed methods, the composition comprises one or more bacteria.

In certain embodiments of the claimed methods, the bacterial species is detected or quantitated by means of DNA sequencing, RNA sequencing, protein sequence homology or other biological marker indicative of the bacterial species.

In certain embodiments of the claimed methods, the sample is a faecal sample, or a sample from the gastrointestinal tract. In certain embodiments of the claimed methods, the sample is a ileal, jejunal, duodenal or colonic sample.

In certain embodiments of the claimed methods, the feline is a cat.

### BRIEF DESCRIPTION OF DRAWINGS

FIG. 1A: Bacterial taxa (OTUs) ranked by importance scores describing consistency in assignment of samples to the appropriate feline study group (adult, senior and geriatric cats).

FIG. 1B: Cumulative sensitivity and positive predicted values for successively increasing numbers of feline OTUs ranked on order of importance in predictive model.

FIG. 1C: Accuracy plot for successively increasing numbers of OTUs ranked in importance order.

FIG. 2A: PLS correlation plot of abundance data for all 113 OTUs and the rare taxa group. Sample and OTU descriptors have been removed for ease of visualisation and replaced with general descriptors for orientation purposes. Faeces samples are represented in individual horizontal rows and are clustered according to likeness while bacterial OTUs are represented by individual columns within the heat plot. Colour coding is indicative of the degree and direction of correlation.

FIG. 2B: Example PLSDA correlation plot for clustering of the relative abundance data of control cats and test samples using the 27 taxa most influential in detecting biological age of the faecal microbiota. Samples are represented on the left hand y axis by group (red, adult; green, senior and blue geriatric). Faeces samples are represented in individual horizontal rows (described on the right hand vertical axis) and are clustered according to likeness, while bacterial OTUs are represented by individual columns within the heat plot. Colour coding of the points within the correlation plot is indicative of the degree and direction of correlation.

FIG. 3: Shannon diversity of the faecal microbiota in adult cats by life stage group (*p=0.007).

FIG. 4 corresponds to Table 1.1, which provides the bacterial species that can be used to assign the cat microbiome to different life stages (kitten, youth, adult, senior, and geriatric).

FIG. 5 corresponds to Table 1.2, which provides the 16S rDNA sequences of the bacterial species provided in FIG. 4 (Table 1.1).

FIG. 6 corresponds to Table 1.3, which provides the accuracy with which bacterial species can be used to assign the cat microbiome to different life stages.

FIG. 7 corresponds to Table 2.1, which provides the bacterial species that can be used to assign the cat microbiome to the adult, senior, and geriatric life stages.

FIG. 8 corresponds to Table 2.2, which provides the 16S rDNA sequences of the bacterial species provided in FIG. 7 (Table 2.1).

FIG. 9 corresponds to Table 3.1, which provides the Shannon diversity in the microbiota of adult, senior, and geriatric cats.

### DETAILED DESCRIPTION

### The Microbiome Age Status

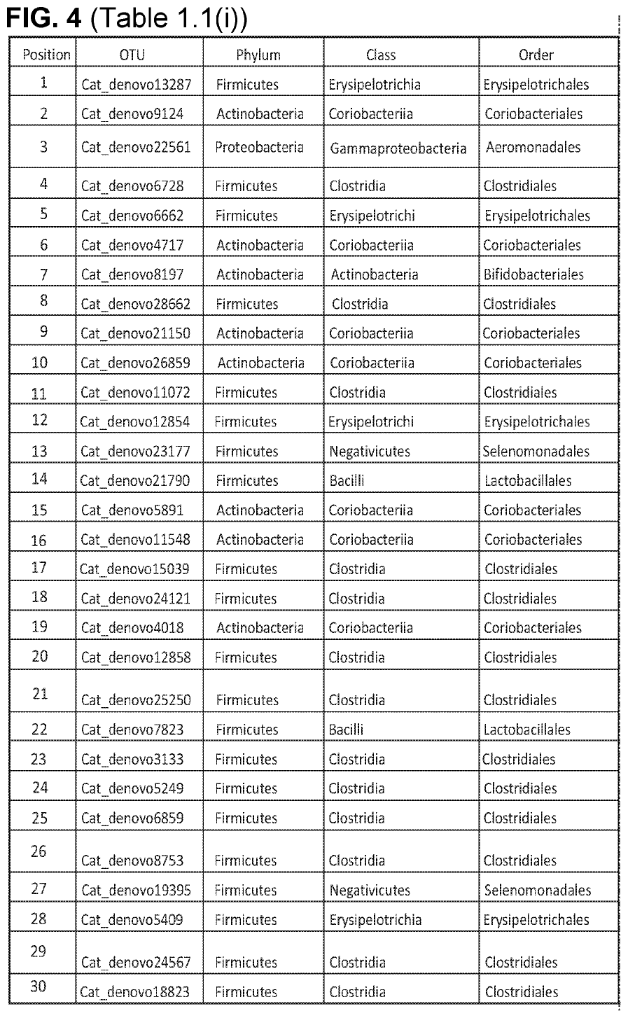

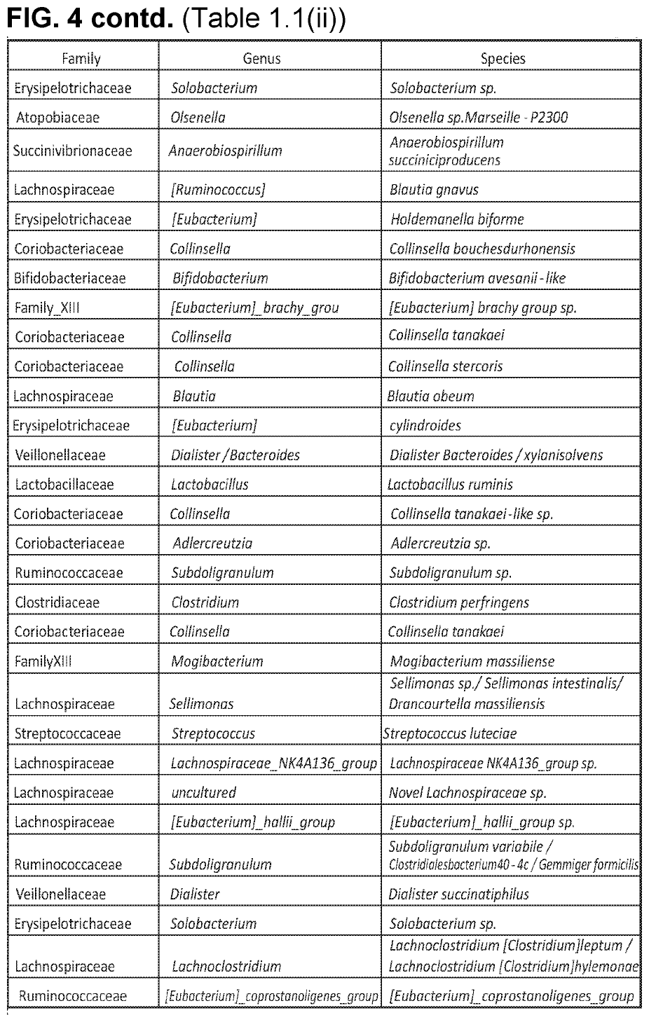

The methods of the present disclosure can be used to identify the age status of the microbiome from a feline. The microbiome can be assigned to a particular “age” or “lifestage” or “life stage” by comparing the bacterial species in the sample, and optionally their relative abundance, to a control data set. Generally, the control data set will represent the microbiome of a feline with known microbiome composition and diversity ranges for the feline's age. For example, the microbiome composition of a number of felines from a specific age group (kitten, youth, adult, senior, geriatric) can be analysed. In addition, or alternatively, the microbiome composition can be analysed from the same feline at intervals within a life stage or in different life stages. Bacterial species which show a high specificity for the different age groups across all tested individuals can then be used as a control data set. The control data set can also be based on the analysis of a single individual, for example at different life stages. In particular embodiments, the data set shown in FIG. 4, 6, or 7 (Tables 1.1, 1.3 or 2.1) serve as a control data set in the methods of the present disclosure.

In some embodiments, the microbiome age status is determined by comparing the microbiome in the sample to the microbiome composition with a known microbiome age status (control microbiome) and determining the microbiome age status based on the similarity to the control. Thus, in some embodiments, the control data set can comprise typical microbiomes of felines at different life stages. In particular embodiments, the control data set comprises microbiome data from a kitten, a youth, an adult feline, a senior feline and a geriatric feline. The microbiome age status can be determined by identifying the control microbiome to which the microbiome of the feline has the most similarity and assigning the corresponding life stage to the feline. These microbiome data can be obtained by the methods discussed above.

In some embodiments, one or a few bacterial species (e.g., less than 5, 7, 8, 10, 15, 20, 25, 27 or 30) are detected and/or quantitated. In a particular embodiment, a minimum of two bacterial species are analysed as the presently disclosed subject matter has shown that such methods have good sensitivity. In other embodiments, a minimum of 7, or exactly 7 bacterial species are analysed, as the presently disclosed subject matter has shown that such methods have peak accuracy in assigning samples to appropriate life stages. These will generally be bacteria which allow an unambiguous allocation to one (or possibly two) of the different life stages. In these embodiments, the one or more bacterial species that are assessed are based on a data set which shows that these are indicative of a certain life stage. Thus, in these embodiments, the detection of the species of interest and the subsequent assignment of the microbiome age status based on these data constitutes correlating the species in the sample to a control data set.

The presently disclosed subject matter has discovered that certain bacterial species are highly indicative of the age status of the feline's microbiome. The bacterial species shown in FIG. 4 (Table 1.1) have high consistency in correctly assigning samples to cats at different life stages (kitten, youth, adult, senior and geriatric). In some embodiments, the methods of the present disclosure thus allow the determination of the microbiome age status of an adult, senior or geriatric feline. The results can then be assessed to determine whether the microbiome age status concurs with the feline's actual age. If not, steps to shift the microbiome age status can be taken, as discussed below. In particular embodiments, the bacterial species which are analysed in the methods of the present disclosure are quantitated. The presently disclosed subject matter has discovered that the relative abundance of different bacterial species within the gut and the faecal microbiome shifts as the cat ages, as discussed in detail in the examples below. The combination and abundance of the bacterial species detected within the microbiome can therefore allow the determination of the age status of the microbiome in a cat/feline, as discussed in detail in the examples below.

The methods of the present disclosure generally detect and/or quantitate one or more different bacterial species, for example one or more bacterial genera and/or one or more bacterial species through the detection of gene sequences or other biomarkers.

In addition to those described herein, techniques which allow a skilled person to detect and quantitate bacterial species are well known in the art. These include, for example, polymerase chain reaction (PCR) and quantitative PCR 16S rDNA amplicon sequencing, shotgun sequencing, metagenome sequencing, Illumina sequencing, and nanopore DNA sequencing techniques. In particular embodiments, the bacterial species are determined by sequencing the 16S rDNA sequence. Other methods would include shotgun sequencing to determine characteristic whole genome gene sequences or other methods such as spectrometry for detection of metabolites and a range of methods for biomarker detection for identification of the species.

In some embodiments, the bacterial species are determined by sequencing the V4-V6 region, for example using Illumina DNA sequencing techniques. These methods can use the primers 319F: CAAGCAGAAGACGGCATACGAGATGTGACTGGAGTTCAGACGTGTGCTCT TCCGATCT (SEQ ID NO: 43) and 806R: AATGATACGG CGACCACCGA GATCTACACT CTTTCCCTAC ACGACGCTCT TCCGATCT (SEQ ID NO: 44).

In further embodiments the bacterial species are be detected by other means known in the art such as, for example, RNA sequencing, protein sequence homology or other biological marker indicative of the bacterial species or the function of the bacterium.

The sequencing data can then be used to determine the presence or absence of different bacterial taxa in the sample. For example, the sequences can be clustered at about 98%, about 99% or 100% identity and bacterial taxa (e.g., abundant taxa representing more than 0.01% or 0.05% of the total sequences) can then be assessed for their relative proportions. Suitable techniques for determining the descriptive nature of bacterial species and biomarkers or combinations of bacteria or biomarkers for their ability to assign a sample to a particular study group such as an age range are known in the art and include, for example, logistic regression analysis; partial least squares discriminate analysis (PLSDA) or random forest analysis and other multivariate methods. The bacterial taxa or biomarkers are then ranked based on their specificity for a particular microbiome age status.

A skilled person will understand that the control data set does not need to be prepared every time a method according to the present disclosure is performed. Instead, a skilled person can analyse a sample as discussed below and compare the results with a database showing the microbial composition of felines with a known healthy microbiome for the age of the animal, or with an average composition and diversity for the life stage or age of the animal.

A further method of the present disclosure is a method of assessing the microbiome age status of a feline to determine whether an intervention (e.g., a treatment) is required, the method comprising (a) quantitating two or more bacterial species in a sample obtained from the feline; (b) determining the relative abundance of said bacterial species; (c) comparing the relative abundance determined in step (b) to that of a control data set; wherein if the comparing of step (c) indicates a difference in microbiome age status to actual age of the feline, an intervention (e.g., a treatment) is recommended. The intervention (e.g., treatment) is one which changes the feline microbiome, as detailed below. The two or more bacterial species can comprise 5 or more, 10 or more, 15 or more, 20 or more, 25 or more or 30 or more bacterial species. The control data set can comprise bacterial species diversity data from a plurality of other felines of the same life stage as the feline tested (such as adult, senior or geriatric), for example from 5 or more, 10 or more, 15 or more, 20 or more, 25 or more, 30 or more 35 or more, or 40 or more other felines.

A further method of the present disclosure is a method of assessing the microbiome age status of a feline to determine whether an intervention (e.g., a treatment) is required, comprising (a) quantitating two or more bacterial species in a sample obtained from the feline; (b) calculating the diversity index; (c) comparing the diversity index to the diversity index of a control data set; wherein if the diversity index calculated in step (b) is less than the 5th percentile or greater than the 95th percentile of the diversity index in a control data set, an intervention (e.g., a treatment) is recommended. The intervention (e.g., treatment) is one which changes the feline microbiome, as detailed below. The two or more bacterial species can comprise 5 or more, 10 or more, 15 or more, 20 or more, 25 or more or 30 or more bacterial species. The control data set can comprise bacterial species diversity data from a plurality of other felines of various life stages, and in some embodiments the same life stage as the feline tested (such as adult, senior or geriatric), for example from 5 or more, 10 or more, 15 or more, 20 or more, 25 or more, 30 or more 35 or more, or 40 or more other felines. Alternatively, the control data set comprises bacterial species diversity data from the same feline at one or more different time points, such as 2, 3, 4 or 5 different time points, in some embodiments, separated by at least 6, 8, 10 or 12 months. Alternatively, the control data set comprises bacterial species diversity data from the same feline prior to injury, illness or stress, such as a result of gastrointestinal upset or travel. In certain embodiments, step (b) comprises calculating the Shannon index diversity by the formula detailed below.

A further method of the present disclosure is a method of assessing the microbiome age status of a feline to determine whether an intervention (e.g., a treatment) is required, comprising (a) quantitating two or more bacterial species in a sample obtained from the feline; (b) determining the relative abundance of said bacterial species; (c) calculating the Shannon index diversity by the formula:

$$Shannon Index ( H ) = - {\sum }_{i = 1}^{s} {p}_{i} ln {p}_{i}$$

wherein if the diversity calculated in step (c) is less than about 2.77 or greater than about 3.99 in an adult feline, less than about 2.41 or greater than about 3.92 in a senior feline, or less than about 1.65 or greater than about 4.17 in a geriatric feline, an intervention (e.g., a treatment) is required. The two or more bacterial species can comprise 5 or more, 10 or more, 15 or more, 20 or more, 25 or more or 30 or more bacterial species. The intervention is one which changes the feline microbiome, as detailed below.

### Bacterial Species

The presently disclosed subject matter has discovered that certain bacteria are particularly useful for determining the microbiome age status of a feline in the methods of the present disclosure. These include bacteria of the genera Solobacterium, Olsenella and Anaerobiospirillum. Particularly useful species within these genera include Solobacterium sp., Olsenella sp. Marseille-P2300 and Anaerobiospirillum succiniciproducens. In some embodiments, the sequence(s) that is detected has at least about 95%, at least about 96%, at least about 97%, at least about 98%, at least about 99% or has 100% identity to the sequence of SEQ ID NOs: 1, 2 and/or 3.

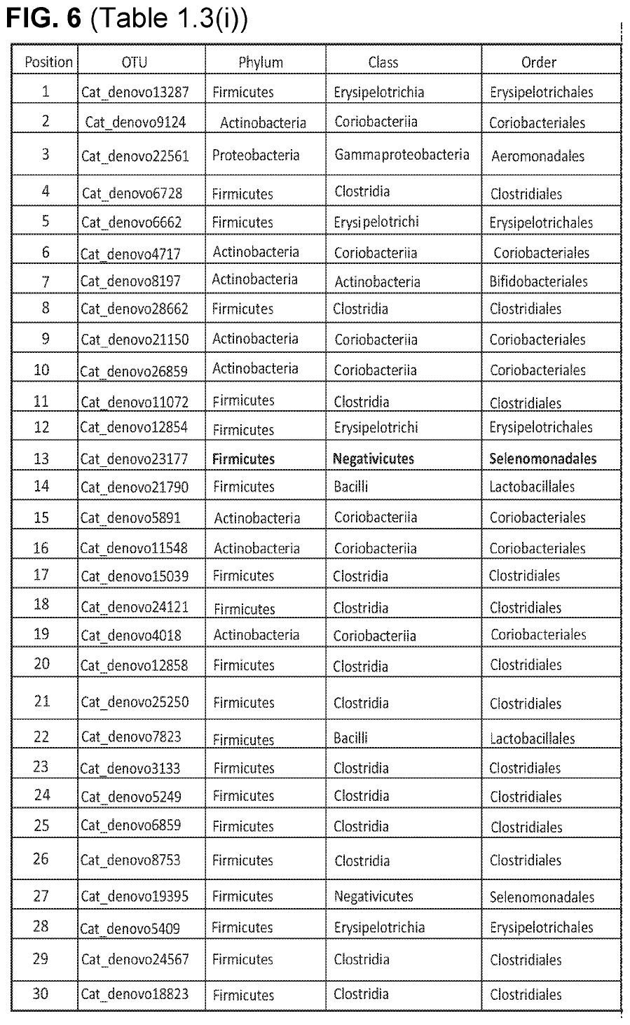

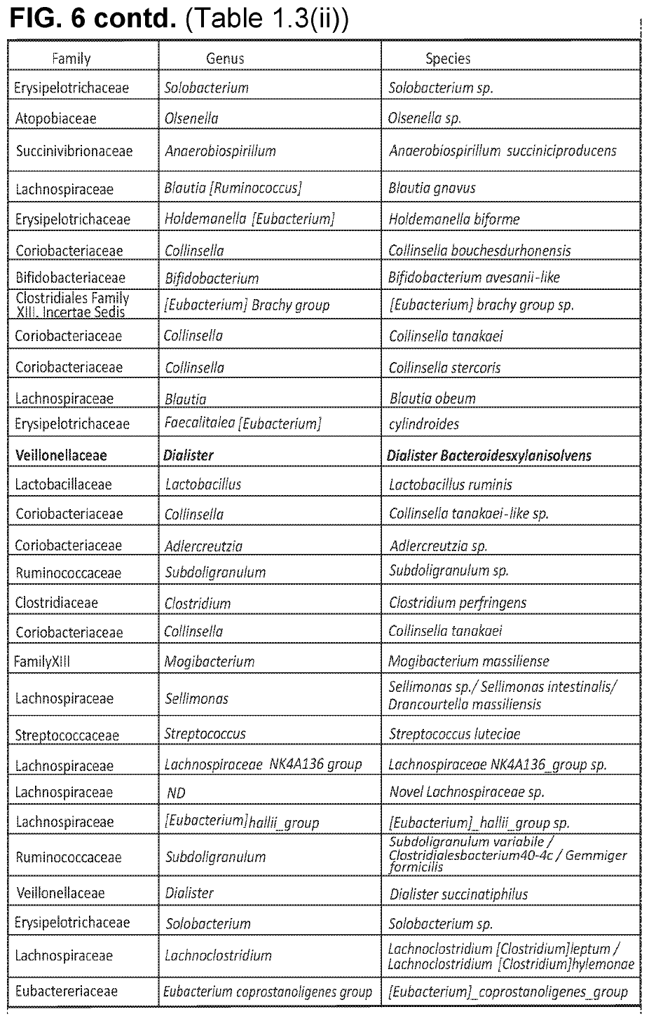

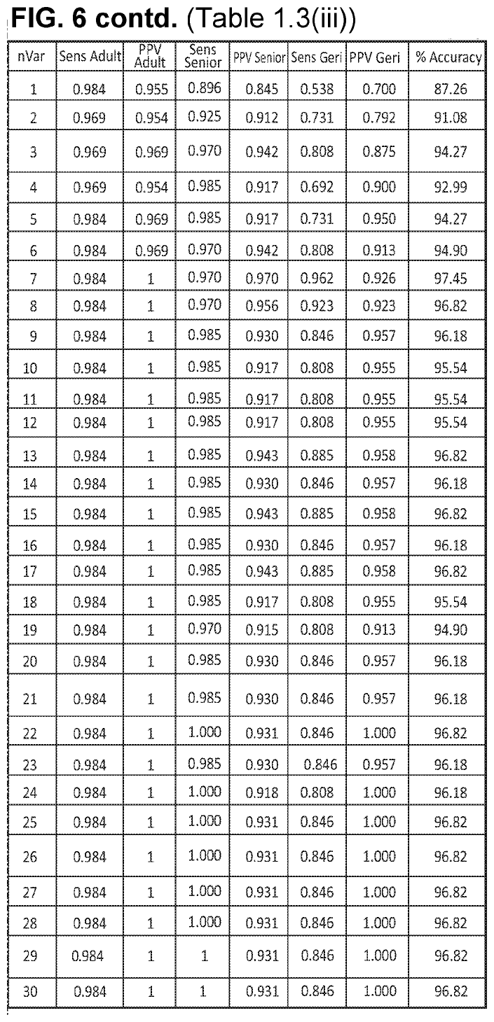

The bacterial species discussed in the preceding paragraph show the highest accuracy in determining a feline's microbiome age status, as shown in FIG. 4 (Table 1.1). The presently disclosed subject matter has further shown that the accuracy increases when more species are detected and/or quantified. Thus, in some embodiments, all of the species mentioned in the preceding paragraph are detected and/or quantified. For example, sequences having at least about 95%, at least about 96%, at least about 97%, at least about 98%, at least about 99% or 100% identity to the sequences of SEQ ID NOs: 1, 2 and 3 are detected and/or quantitated. In further embodiments, in addition to all of the species mentioned in the preceding paragraph, all of the following species are detected and/or quantified: Blautia gnavus, Holdemanella biforme, Collinsella bouchesdurhonensis, and Bifidobacterium avesanii. For example, sequences having at least about 95%, at least about 96%, at least about 97%, at least about 98%, at least about 99% or 100% identity to the sequences of SEQ ID NOs: 4, 5, 6 and 7 are detected and/or quantitated. As shown in FIG. 6 (Table 1.3), peak accuracy in assigning samples to appropriate life stages is achieved when these seven species are detected and/or quantitated. In addition, or alternatively, the methods of the present disclosure also include analysing one or more of the bacterial species and genera discussed below.

Further bacterial species which are useful for distinguishing between an adult, senior, and geriatric microbiomes include species from genera selected from the group consisting of: Solobacterium, Olsenella, Anaerobiospirillum, Blautia, Holdemanella, Collinsella, Bifidobacterium, Eubacterium, Ruminococcus, Dialister, Bacteroides, Lactobacillus, Adlercreutzia, Subdoligranulum, Clostridium, Mogibacterium, Sellimonas, Streptococcus, Subdoligranulum and Lachnoclostridium, or bacterial species from the family Lachnospiraceae. In some embodiments, bacterial species for use in the methods of the present disclosure include Solobacterium sp., Olsenella sp. Marseille-P2300, Anaerobiospirillum succiniciproducens, Blautia gnavus, Holdemanella biforme, Collinsella bouchesdurhonensis, Bifidobacterium sp., Eubacterium brachy, Collinsella tanakaei, Collinsella stercoris, Blautia obeum, Eubacterium cylindroides, Dialister/Bacteroides xylanisolvens, Lactobacillus ruminis, Adlercreutzia sp., Subdoligranulum sp., Clostridium perfringens, Mogibacterium massiliense, Sellimonas sp., Streptococcus luteciae, Lachnospiraceae sp., Subdoligranulum variabile, Dialister succinatiphilus, Lachnoclostridium [Clostridium] leptum, Lachnoclostridium [Clostridium] hylemonae and Eubacterium coprostanoligenes.

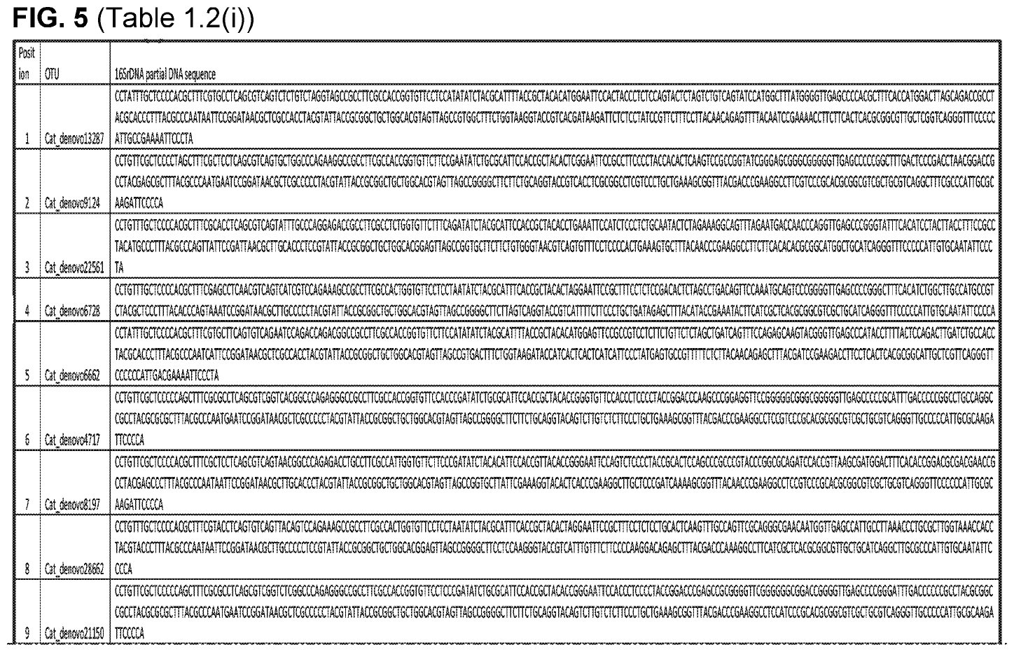

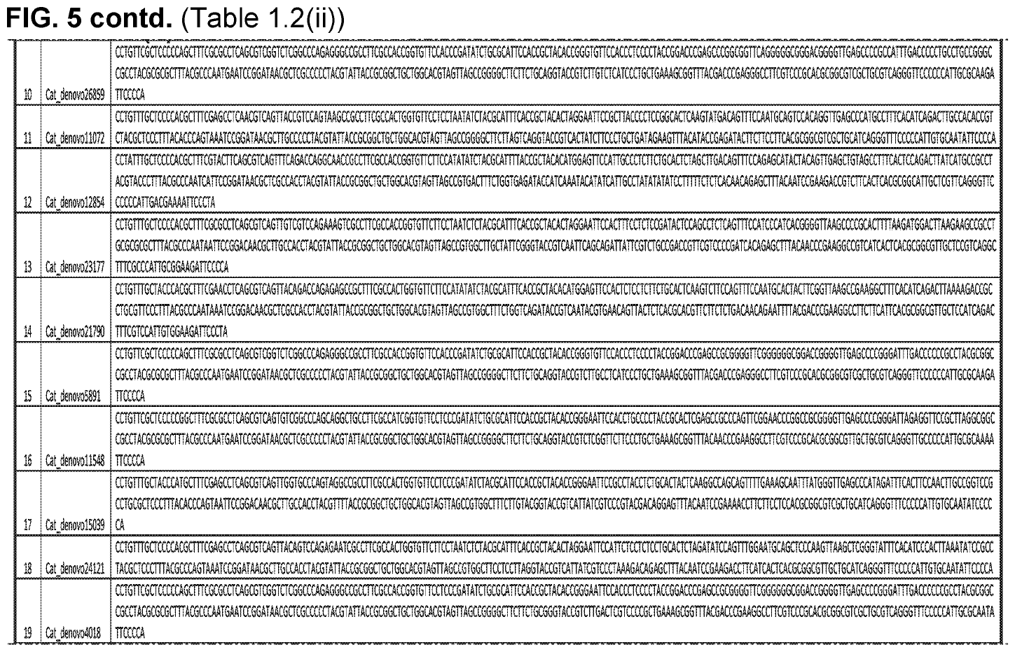

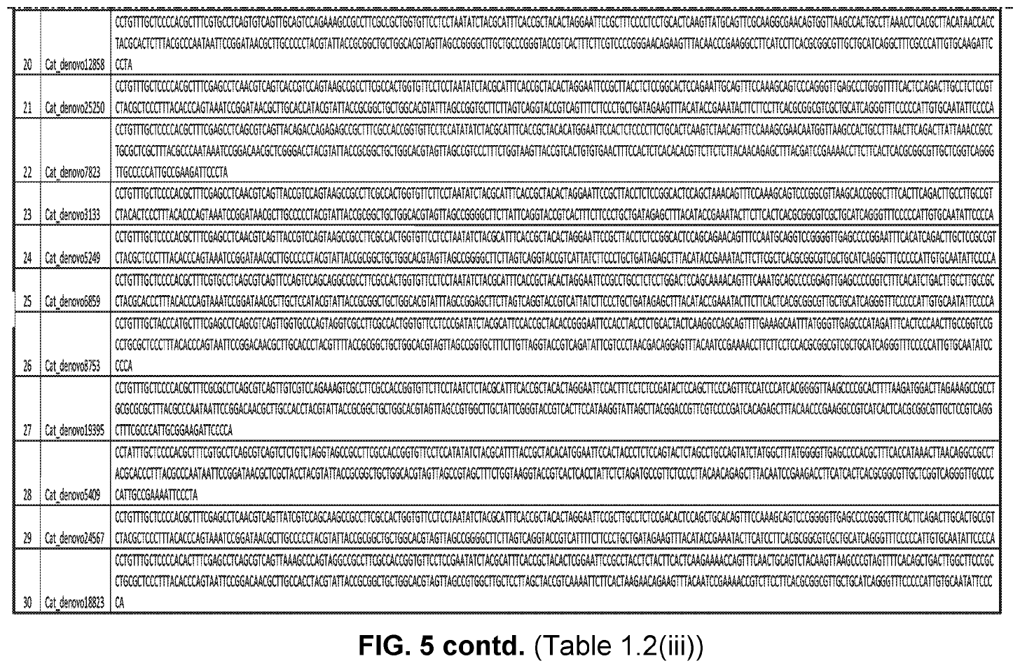

As discussed above and as shown in FIG. 6 (Table 1.3), the accuracy of the methods of the present disclosure increases when more bacterial species are detected and/or quantified. Thus, in some embodiments, more than 5, more than 6, more than 7, more than 8, more than 10, more than 15, more than 20, more than 25 or, all 30 of the bacterial species shown in FIG. 4 (Table 1.1) with the sequences as described in FIG. 5 (Table 1.2) are detected and/or quantified. The bacterial species can be those discussed in the previous paragraphs. For example, in some embodiments, the methods detect and/or quantitate sequences having at least about 95%, at least about 96%, at least about 97%, at least about 98%, at least about 99% or 100% identity to any of the sequences of SEQ ID NOs: 1-30. In particular embodiments, the methods of the present disclosure detect and/or quantitate sequences having at least about 95%, at least about 96%, at least about 97%, at least about 98%, at least about 99% or 100% identity to all of the sequences of SEQ ID Nos: 1-3, or 4-7, or 1-7.

The presently disclosed subject matter has further discovered that bacteria of the genus Solobacterium, are particularly useful for determining the microbiome age status of a feline at the various stages of adult life (adult, senior and geriatric). Therefore, in certain embodiments, the method disclosed herein comprises detecting and/or quantitating a sequence having at least about 95%, at least about 96%, at least about 97%, at least about 98%, at least about 99% or having 100% identity to the sequence of SEQ ID NO: 1.

The presently disclosed subject matter has discovered that bacteria of the genus Olsenella and/or the genus Anaerobiospirillum, in particular the species Olsenella sp. Marseille P2300 and Anaerobiospirillum succiniciproducens are particularly useful for determining the microbiome age status of a feline at the various stages of adult life (youth, adult, senior and geriatric). Thus, in some embodiments, the method disclosed herein comprises detecting and/or quantitating a sequence having at least about 95%, at least about 96%, at least about 97%, at least about 98%, at least about 99% or having 100% identity to the sequences of SEQ ID NO: 2 and/or 3.

Further useful bacterial species include Eubacterium sp., Collinsella sp. Blautia sp., Dialister/Bacteroides sp., Lactobacillus sp., Adlercreutzia sp., Subdoligranulum sp. and particularly Eubacterium brachy, Collinsella tanakaei, Collinsella stercoris, Blautia obeum, Eubacterium cylindroides, Dialister/Bacteroides xylanisolvens, Lactobacillus ruminis, Adlercreutzia sp. and Subdoligranulum sp. In some instances, the method also comprises detecting and/or quantitating a sequence having at least about 95%, at least about 96%, at least about 97%, at least about 98%, at least about 99% or having 100% identity to the sequences of SEQ ID NOs: 8-17.

As shown in FIG. 6 (Table 1.3), the presently disclosed subject matter has found that the specificity increases if more than one bacterial species is detected and/or quantified, with peak specificity achieved with seven species. Thus, in some embodiments, two or more bacterial species (e.g., bacterial genera and/or bacterial species) are detected and/or quantified. The specificity of the method increases as more bacterial species are detected and/or quantified. The methods of the present disclosure can comprise, for instance, detection of 5 or more, 6 or more, 7 or more, 15 or more, 20 or more, or 25 or more or all 30 of the bacterial species described in FIG. 4 (Table 1.1). In some embodiments, the method can comprise detecting all of the bacterial species, or all of the bacterial genera or bacterial species identified in FIG. 4 (Table 1.1) and described by DNA sequences in FIG. 5 (Table 1.2). For example, the methods can comprise detecting sequences having at least about 95%, at least about 96%, at least about 97%, at least about 98%, at least about 99% or having 100% identity to the sequences of SEQ ID NOs: 1-30. In particular embodiments, 7 or more, or specifically exactly 7 bacterial species are detected and/or quantified, as the presently disclosed subject matter has shown that such methods have peak accuracy in assigning samples to appropriate life stages (see FIG. 6 (Table 1.3)).

Where a sequence is detected having at least about 95%, at least about 96%, at least about 97%, at least about 98%, or at least about 99%, or 100% identity to the DNA sequences identified herein, the sequence can stem from a bacterium which is either of the same species or a closely related species. For example, where a sequence has 95% identity to the sequence of SEQ ID NO: x, the sequence will preferably stem from a bacterium in the same family, more preferably the same genus as the bacterium from which SEQ ID NO: x was obtained or even preferably be from the same species. For example, in the case of SEQ ID NO: 6, the bacterium would preferably be of the genus Collinsella and most preferably of the species Collinsella bouchesdurhonensis.

In particular embodiments, wherein the step of correlating the one or more bacterial species in the sample to a control data set comprises the use of partial least squares discriminate analysis (“PLS-DA embodiment”), detection and/or quantitation of different bacterial species can be useful (as demonstrated in Example 2). In this embodiment, useful genera include Solobacterium, Eubacterium, Blautia, Lachnoclostridium, Olsenella, Acidaminococcus, Anaerobiospirillum, Megasphaera, Bifidobacterium, Coriobacteriaceae, Catenibacterium, Drancourtella, Sellimonas, Ruminococcus, Dorea, Collinsella, Holdemanella, Mogibacterium and Anaerostipes; useful species include Solobacterium sp., Eubacterium brachy, Blautia obeum, Lachnoclostridium [Clostridium] leptum, Lachnoclostridium [Clostridium] hylemonae, Olsenella sp., Acidaminococcus fermentans, Acidaminococcus timonensis, Anaerobiospirillum succiniciproducens, Megasphaera indica, Megasphaera elsdenii, Megasphaera sp. Bifidobacterium gallinarum, Bifidobacterium pullorum, Bifidobacterium saeculare, Bifidobacterium subtile, Coriobacteriaceae sp., Bifidobacterium sp., Catenibacterium mitsuokai, Drancourtella massiliensis, Sellimonas intestinalis, Ruminococcus sp, Lachnospiraceae sp., Dorea sp., Blautia producta, Coriobacteriaceae sp, Collinsella sp., Holdemanella biforme, Mogibacterium massiliense, Anaerostipes hadrus, Anaerostipes sp., Blautia gnavus, Collinsella stercoris/intestinalis, Bifidobacterium sp. and Collinsella tanakaei. Such methods can involve detection and/or quantitate sequences having at least about 95%, at least about 96%, at least about 97%, at least about 98%, at least about 99% or 100% identity to any of the sequences of SEQ ID Nos: 1, 8, 11, 29, 2, 31, 3, 32, 33, 34, 35, 36, 37, 38, 21, 28, 39, 40, 41, 9, 5, 20, 42, 4, 10, 7 and 15.

The PLS-DA embodiments include detection or quantitation of a bacterial species from the genus Solobacterium, and more specifically also the species Eubacterium brachy and Blautia obeum, for example by detecting and/or quantitating a 16S rDNA in the bacterial species having at least about 95%, at least about 96%, at least about 97%, at least about 98%, at least about 99%, or 100% identity with the sequence of SEQ ID NO: 1, and also SEQ ID NO: 8 and 11. In further embodiments, the method comprises detecting or quantitating Lachnoclostridium [Clostridium] leptum/Lachnoclostridium [Clostridium] hylemonae, Olsenella sp., Acidaminococcus fermentans/Acidaminococcus timonensis, and Anaerobiospirillum succiniciproducen, for example by detecting and/or quantitating a 16S rDNA with at least about 95%, at least about 96%, at least about 97%, at least about 98%, at least about 99%, or 100% identity with the sequence of SEQ ID NOs: 29, 2, 31 and 3. In further embodiments, the method comprises detecting or quantitating Megasphaera sp., Bifidobacterium sp., Coriobacteriaceae sp., Catenibacterium sp., Drancourtella sp., Sellimonas sp., Ruminococcus sp. Dorea sp., Collinsella, Holdemanella, Mogibacterium and Anaerostipes; particularly detecting or quantitating Megasphaera indica/Megasphaera elsdenii, Megasphaera sp., Bifidobacterium gallinarum/Bifidobacterium pullorum/Bifidobacterium saeculare/Bifidobacterium subtile, Coriobacteriaceae sp., Bifidobacterium sp., Catenibacterium mitsuokai, Drancourtella massiliensis/Sellimonas intestinalis/Ruminococcus sp/Lachnospiraceae sp., Dorea sp., Blautia producta, Coriobacteriaceae sp, Collinsella sp., Holdemanella biforme, Mogibacterium massiliense, Anaerostipes hadrus, Anaerostipes sp., Blautia gnavus, Collinsella stercoris/intestinalis, Bifidobacterium sp. and Collinsella tanakaei.

### The Feline

The methods of the present disclosure can be used to determine the microbiome health of a feline. This genus comprises species in the Felidae family. These species include African-Asian Wildcat (Felis silvestris ornata), African Golden Cat (Profelis aurata), Andean Mountain Cat (Leopardus jacobita), Asiatic Golden Cat (Catopuma temminckii), Bay Cat (Catopuma badia), Black-footed Cat (Felis nigripes), Bobcat (Lynx rufus), Bornean Clouded Leopard (Neofelis diardi), Canadian Lynx (Lynx canadensis), Caracal (Caracal caracal), Cheetah (Acinonyx jubatus), Chinese Desert Cat (Felis bieti), Clouded Leopard (Neofelis nebulosa), Domestic Cat (Felis catus), Eurasian Lynx (Lynx lynx), European Wildcat (Felis silvestris), Fishing Cat (Prionailurus viverrinus), Flat-headed Cat (Prionailurus planiceps), Geoffroy's Cat (Leopardus geoffroyi), Iberian Lynx (Lynx pardinus), Iriomote Cat (Prionailurus iriomotensis), Jaguar (Panthera onca), Jaguarundi (Herpailurus yagouarundi), Jungle Cat (Felis chaus), Kodkod (Leopardus guigna), Leopard Cat (Prionailurus bengalensis), Leopard (Panthera pardus), Lion (Panthera leo), Marbled Cat (Pardofelis marmorata), Margay (Leopardus wiedii), Mountain Lion (Puma concolor), Ocelot (Leopardus pardalis), Oncilla (Leopardus tigrinus), Pallas's Cat (Otocolobus manul), Pampas Cat (Leopardus colocolo), Rusty-spotted Cat (Prionailurus rubiginosus), Sand Cat (Felis margarita), Serval (Leptailurus serval), Snow Leopard (Uncia uncia), and Tiger (Panthera). In some embodiments, the feline is a domestic cat, herein referred to as a cat.

Furthermore, in some embodiments, the feline is healthy. “Healthy,” as used herein, refers to a feline who has not been diagnosed with a disease that is known to affect the microbiome. Examples of such diseases include, but are not limited to, irritable bowel syndrome, ulcerative colitis, Crohn's and inflammatory bowel disease. In a particular embodiment, the feline does not suffer from dysbiosis. Dysbiosis refers to a microbiome imbalance inside the body, resulting from an insufficient level of keystone bacteria (e.g., bifidobacteria, such as B. longum subsp. infantis) or an overabundance of harmful bacteria in the gut. Methods for detecting dysbiosis are well known in the art.

One advantage of the methods of the present disclosure is that they allow a skilled person to determine whether the feline's microbiome is healthy, taking into account the feline's life stage.

There are numerous different breeds of cats. A summary of the different life stages is provided in Table 1.0 below.

TABLE 1.0

| Youth | Adult | Senior | Geriatric |
|---|---|---|---|
| 1 to 4 years | 5 to 9 years | 10 to 13 years | ≥14 years |

A skilled person will appreciate that the age ranges discussed above will not always strictly apply to each individual feline. Rather, a skilled person will be able to categorise a feline into a specific life stage by its physiological features, for example.

### The Sample

According to the methods of disclosed herein, the microbiome composition of a feline is analysed by analysing a sample taken from the feline. The sample can be a faecal sample or a sample from the gastrointestinal lumen of the feline. Faecal samples are convenient because their collection is non-invasive and it also allows for easy repeated sampling of individuals over a period of time. However, other samples can also be used with the methods disclosed herein, including, but not limited to, ileal, jejunal, duodenal samples and colonic samples.

In some embodiments, the sample is a fresh sample. In further embodiments, the sample is frozen or stabilised by other means, such as addition to preservation buffers, or by dehydration using methods such as freeze drying, before use in the methods of the present disclosure.

Before use in the methods of the present disclosure, in some embodiments, the sample is processed to extract DNA. Methods for isolating DNA are well known in the art, as reviewed in reference [8], for example. Suitable methods include, for instance, the QIAamp Power Faecal DNA kit (Qiagen).

### Changing the Microbiome

In some embodiments, the methods of the present disclosure comprises a further step of changing the composition of the microbiome. The composition of the microbiome can be changed by administering to the feline a dietary change, a functional food, a supplement, a nutraceutical, or a pharmaceutical composition that is capable of changing the composition of the microbiome. Such functional foods, nutraceuticals, live biotherapeutic products (LBPs) and pharmaceutical compositions are well known in the art and comprise bacteria [9]. They can comprise single bacterial species selected from the group consisting of Bifidobacterium sp. such as B. animalis (e.g., B. animalis subsp. animalis or B. animalis subsp. lactis], B. bifidum, B. breve, B. longum (e.g., B. longum subsp. infantis or B. longum subsp. longum), B. pseudolongum, B. adolescentis, B. catenulatum, or B. pseudocatanulatum, single bacterial species of Lactobacillus, such as L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. salivarius, L. paracasei, L. kisonensis., L. paralimentarius, L. perolens, L. apis, L. ghanensis, L. dextrinicus, L. shenzenensis, L. harbinensis, or single bacterial species of Pediococcus, such as P. parvulus, P. lolii, P. acidilactici, P. argentinicus, P. claussenii, P. pentosaceus, or P. stilesii or similarly species of Enterococcus such as E. faecium or Bacillus species such as Bacillus subtilis, B. coagulans B. indicus or B. clausii. Additionally, the methods can include combinations of these and other bacterial species. The amount of the dietary change, the functional food, the supplement, the nutraceutical composition, or the pharmaceutical composition that is administered to the feline can be an amount that is effective to effect a change in the composition of the microbiome.

The further step of changing the composition of the microbiome can be performed in instances where a feline's microbiome age status does not positively concur with its actual age. For example, the methods of the present disclosure can reveal that an adult cat has a microbiome composition and diversity representative of a senior or geriatric cat. As discussed above, characteristics associated with the adult microbiome are considered the healthiest microbiome characteristics and so in these circumstances it would be highly desirable in the older cat to make a dietary change and/or to administer a functional food, nutraceutical, LBP or pharmaceutical composition to shift the microbiome back to an adult microbiome composition/status.

Similarly, in other embodiments, it can be desirable to shift the microbiome so that the microbiome age status does not concur with the feline's actual age. For example, an older cat in the senior or geriatric life stage can benefit from receiving a diet change, functional food, nutraceutical, LBP or pharmaceutical composition to shift the microbiome to one representative of an adult cat, particularly if suffering from recurrent diarrhea or other microbiome related condition.

The methods of the present disclosure can also be used to assess the success of a treatment as described above. To this end a feline can receive a change in diet, functional food, supplement, LBP, nutraceutical or pharmaceutical composition which is capable of changing the composition of the microbiome. Following administration of the treatment through nutrition (for example after about 1 day, 2 days, 5 days, 1 week, 2 weeks, 3 weeks, 1 month, 3 months, 6 months etc.), the microbiome age status can be assessed using any of the methods of the present disclosure. In certain embodiments, the microbiome age status is determined before and after the dietary change or the administration of the functional food, supplement, LBP, nutraceutical or pharmaceutical composition.

### Monitoring

In some embodiments, the methods of described herein are performed once to determine a feline's microbiome age status and compare to chronological age. The methods of the present disclosure can also be performed more than once, for example two times, three times, four times, five times, six times, seven times or more than seven times. This allows the microbiome age status to be monitored over time. This can be useful for example where a feline is receiving treatment to shift the microbiome or is at an age representing a transition between life stages to track the need for an intervention in the future. The first time the method is performed the microbiome age status is determined and, following an intervention such as a dietary change or administration of a supplement, functional food, nutraceutical or pharmaceutical composition, the method is repeated to assess the influence of the composition or intervention on the microbiome. The microbiome age status can also be determined for the first time after the feline has received treatment and the method repeated afterwards to assess whether there is a change in the microbiome age status.

The methods described herein can be repeated several days, about one week, about two weeks, about three weeks, about one month, about two months, about three months, about four months, about five months, about six months, about 12 months, about 18 months, about 24 months, about 30 months, about 36 months, or more than about 36 months apart.

### Treatment

In some embodiments, the methods of the present disclosure can also relate to methods for treating a feline. The feline can have a microbiome age that does not concur with its actual age, e.g., an adult cat having a microbiome composition and diversity of a senior or geriatric cat. In other embodiments, the methods of the present disclosure can relate to methods for treating a feline to shift the microbiome age of the feline to be lower than the feline's actual age. These methods for treating include (i) identifying the feline as requiring treatment by determining the microbiome age according to any of the methods disclosed herein, and (ii) administering to the feline a dietary change, a functional food, a supplement, a nutraceutical, or a pharmaceutical composition as disclosed herein that is capable of changing the composition of the microbiome. The amount of the dietary change, the functional food, the supplement, the nutraceutical composition, or the pharmaceutical composition that is administered to the feline can be an amount that is effective to effect a change in the composition of the microbiome, or to improve any symptoms relating to the feline having an unhealthy microbiome status. Optionally, in some embodiments, the method further includes determining the microbiome age following the administration of the dietary change, the functional food, the supplement, the nutraceutical, or the pharmaceutical composition to evaluate the effectiveness of the treatment.

### General

The practice of the present disclosure will employ, unless otherwise indicated, conventional methods of chemistry, biochemistry, molecular biology, immunology and pharmacology, within the skill of the art. Such techniques are explained fully in the literature. See, e.g., references [10-17], etc.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref [18]. A particular alignment is determined using the BLAST (basic local alignment search tool) algorithm or the Smith-Waterman homology search algorithm using an affine gap search with a gap open penalty of 12 and a gap extension penalty of 2, BLOSUM matrix of 62. The Smith-Waterman homology search algorithm is disclosed in ref [19]. The alignment can be over the entire reference sequence, i.e., it can be over 100% length of the sequences disclosed herein.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “containing,” and “comprising” are interchangeable and one of skill in the art is cognizant that these terms are open ended terms. Further, the term “comprising” encompasses “including” as well as “consisting,” e.g., a composition “comprising” X can consist exclusively of X or can include something additional, e.g., X+Y.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, alternatively up to 10%, alternatively up to 5%, and alternatively still up to 1% of a given value. Alternatively, particularly with respect to biological systems or processes, the term can mean within an order of magnitude, alternatively within 5-fold, and alternatively within 2-fold, of a value. In certain embodiments, the term “about” in relation to a numerical value x is optional and means, for example, x+10%.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition (e.g., a dietary change, a functional food, a nutraceutical composition, or a pharmaceutical composition) to change the composition of a microbiome in a feline having an unhealthy microbiome, the effective amount is an amount sufficient to bring the health status of the microbiome back to a healthy state, which is determined according to one of the methods disclosed herein. In certain embodiments, an effective treatment as described herein can also include administering a treatment in an amount sufficient to decrease any symptoms associated with an unhealthy microbiome. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of symptoms of an unhealthy microbiome. An effective amount can be administered in one or more administrations. A likelihood of an effective treatment described herein is a probability of a treatment being effective, i.e., sufficient to alter the microbiome, or treat or ameliorate a digestive disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. In certain embodiments, the decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment.

The word “substantially” does not exclude “completely” e.g. a composition which is “substantially free” from Y can be completely free from Y. Where necessary, the word “substantially” can be omitted from the definition of the present disclosure.

Unless specifically stated, a process or method comprising numerous steps can comprise additional steps at the beginning or end of the method, or can comprise additional intervening steps. Also, steps can be combined, omitted or performed in an alternative order, if appropriate.

Various embodiments of the methods of the present disclosure are described herein. It will be appreciated that the features specified in each embodiment can be combined with other specified features, to provide further embodiments. In particular, embodiments highlighted herein as being suitable, typical or preferred can be combined with each other (except when they are mutually exclusive).

### EXAMPLES

The presently disclosed subject matter will be better understood by reference to the following Example, which is provided as exemplary of the invention, and not by way of limitation.

### Overview

A cross-sectional analysis of the faecal microbiota was conducted in a cohort of 48 cats aged between 4.7 and 16.2 years at the Mars Inc. Pet Health and Nutrition Centre (PHNC, Lewisburg, Ohio). Animals were assigned to one of 3 groups defined as different life stages including adult (target age range 3-6 years), senior (target age range 9.5-12 years) and geriatric (target age range 14+ years) cats. Group assignment was based on age with specific categories guided by the findings of the research on evidence-based analysis of Banfield veterinary diagnoses with age in cats and dogs (Salt and Saito, personal communication). All cats were fed a consistent diet for a period of 30 days with fresh faecal samples collected at days 21, 24 and 28 (+/−2 days).

The cohort of 48 cats comprised 20 adult cats (mean age 5.66 years; 8 male; 12 female), 20 senior cats (mean age 10.10 years; 10 male; 10 female) and 8 geriatric cats (mean age 14.78 years; 3 male; 5 female). Differences in a range of bacterial species with diverse nutritional characteristics were observed in the microbiota of cats in different life stages, suggestive that changes occur in the feline microbiome with age. Significant contrasts in the relative abundance was observed between life stage groups for more than 20% (25 of 113) of the abundant bacterial species even after 21 days of consistency in dietary intake. A molecular fingerprint characteristic of life stage was identified based on the relative abundance of 27 bacterial species in feline faeces.

These insights provide putative signatures of an aging feline microbiome that can be leveraged in the development of microbiome health monitoring tools and dietary interventions towards maintaining a healthy microbiome throughout life.

### Materials and Methods

### Animals

The cohort comprised 20 adult cats (aged 4.7-6.8 years), 20 senior cats (aged 8.1-12.5 years) and 8 geriatric (aged 12.6-16.2 years) were recruited to the study. All animals received a veterinary health check to determine suitability for inclusion prior to the start of the study. Cats were provided with access to fresh drinking water at all times and were exercised consistently throughout the study as per standard practice for PHNC. All cats were involved in their normal daily activities throughout the study and received their standard medication as required. Cats were familiarised to study personnel and were socialised for a minimum of 1 hour each day following the standard PHNC care package.

Diet The cats were fed the same commercially available nutritionally complete diet (Royal Canin indoor 7+ dry cat food) for a period of 30 days. Additionally, a 10 g bolus of RC wet cat food was fed daily across the cohort to facilitate feeding of medication in those cats with active veterinary prescriptions. Cats were fed at energy levels (mean energy requirements; MER) to maintain a healthy body condition score (BCS) and bodyweight (within +/−5%) throughout the study. Two equal food portions were offered (˜50% MER) twice a day.

### Data Collection

During the study the following co-variates were collected for inclusion in data analyses to establish whether differences in the microbiome were associated with adult, senior and geriatric life stages. Animal housing details; Daily food intake; Bodyweight and body condition score and daily and overnight faeces scores per room. Faeces were scored using the WALTHAM 17-point faeces quality scale and incidences of poor faeces (outside of the acceptable range 1.5-3.75) were recorded.

### Faeces Sample Collection, Processing and Analysis

Fresh faecal samples were collected no more than 15 minutes after defecation. Following collection, faeces were portioned into aliquots and stored at −80 degrees centigrade prior to processing for DNA extraction using the QIAamp Power Faecal DNA kit (Qiagen). DNA concentrations achieved per sample were determined by standard nanodrop DNA quantification methods. PCR amplification was conducted on individual samples to generate dual indexed, barcoded 16SrDNA sequencing libraries suitable for analysis on the Ilumina MiSeq sequencing system. DNA sequencing was conducted by Eurofins Applied Genomics Laboratory (Eurofins Genomics; Anzinger Str. 7a; 85560 Ebersberg; Germany. Samples were quantified and pooled prior to loading, library pool concentrations were determined prior to processing to optimise Ilumina channel loading. Quality thresholds of a minimum of 1,000 sequence reads per sample were defined for sequence data, which was de-noised and clustered based on percentage identity (98.5%) using the WALTHAM bioinformatics analysis pipeline. The resulting operational taxonomic unit (OTU) data was analysed to determine whether differences in the detection (presence/absence) or relative abundance of species were observed between groups.

### Statistical Methods

Following DNA extraction from the faecal sample and Illumina sequencing of the V4-V6 region using DNA oligonucleotide primers (sequence (319F: CAAGCAGAAG ACGGCATACG AGATGTGACT GGAGTTCAGA CGTGTGCTCT TCCGATCT (SEQ ID NO: 43) and 806R: AATGATACGG CGACCACCGA GATCTACACT CTTTCCCTAC ACGACGCTCT TCCGATCT (SEQ ID NO: 44)) the resulting DNA sequences were clustered at 98% identity and abundant species (representing >0.001 of the total sequences) were then assessed for their relative proportions. Preliminary exploratory analyses were performed using principal components analysis (PCA) and t-distributed stochastic neighbour embedding (t-SNE) to reduce the dimension of the data and visually represent groups.

Assessment of Individual Microbiome Elements with Life Stage/Age:

Shannon diversity was calculated for each sample and modelled using a linear mixed effects model with a fixed effect of age group and random intercept of pet. Pairwise comparisons of the life stage groups were performed with a controlled familywise error rate of 5%.

Prior to individual modelling of the bacterial OTUs (representing individual species) rare OTUs were combined to create a single ‘rare’ group. A rare OTU/group was identified as those with a mean proportion of less than 0.05% and not present in at least two samples from a single age group. Clustered OTUs represented by an individual representative (reference) 16SrDNA sequence were analysed individually using generalised linear mixed effects models (GLMM) with a binomial distribution and logit link function. In the models counts and total counts represented the response variables using age group as a fixed effect, and a random intercept of dog to account for the repeated measurements. All pairwise comparisons were performed between age groups using a permutation test permuting the age group indicator for each pet. A familywise error rate of 5% was maintained using multiple comparisons correction.

The associated primary measures were analysed with linear and generalised linear models, with random effects in the cases where repeated measures were taken per pet. Analysis was conducted both with and without co-housing included as a factor.

Analysis of Gross Microbiome Characteristics with Life Stage/Age:

A supervised dimension reduction and regression method, partial least squares discriminate analysis (PLS) was used to relate the primary measures to the microbiota data and assess clustering of sample likeness characteristics.

Random Forest analysis was conducted to assess the accuracy in assignment of samples to cohort group based on the 16S rDNA gene distribution and abundance within the faecal microbiota. Importance scores were calculated using 2000 random forests each with 2000 trees using the default number of variables per tree (square root of the number of variables) and permutation importance score calculation. From these 2000 random forests the average ranks of each OTU were calculated, with 1 being the most important in determining sample fit to age. Positive predictive value and specificity were calculated by fitting random forests to data with successively more OTUs (taken in importance order).

Analyses were performed using R version 3.5.1 and the ranger library.

### Example 1—A Method of Detecting the Biological Age Status of the Faecal Microbiome in Cats

Random Forest analysis was applied to the data set to assess the accuracy in assignment of samples to cohort group based on the 16S rDNA gene distribution and abundance within the faecal microbiota. Importance scores were calculated using 2000 random forests each with 2000 trees using the default number of variables per tree (square root of the number of variables) and permutation importance score calculation. From these 2000 random forests the average ranks of each OTU were calculated, with 1 being the most important in determining sample fit to age. Positive predictive value and specificity were calculated by fitting random forests to data with successively more OTUs (taken in importance order).

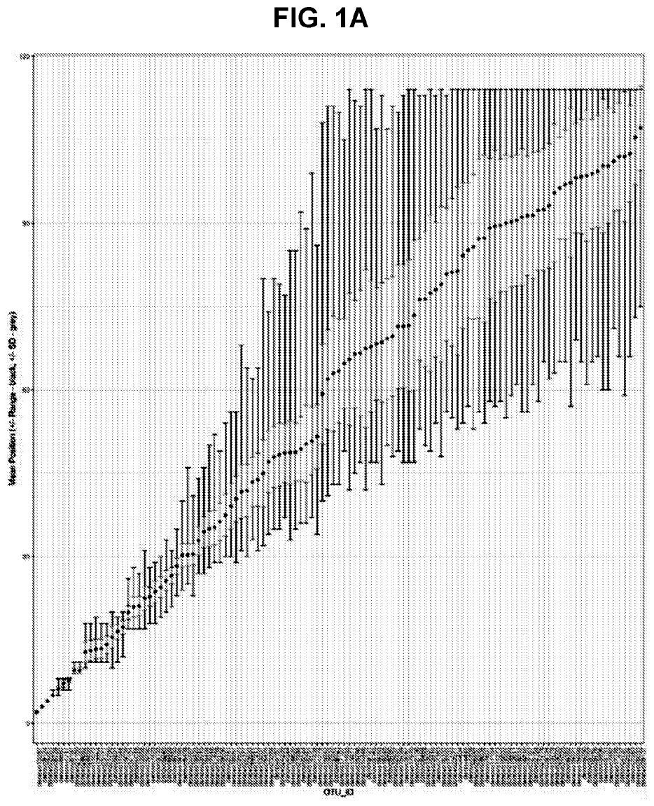

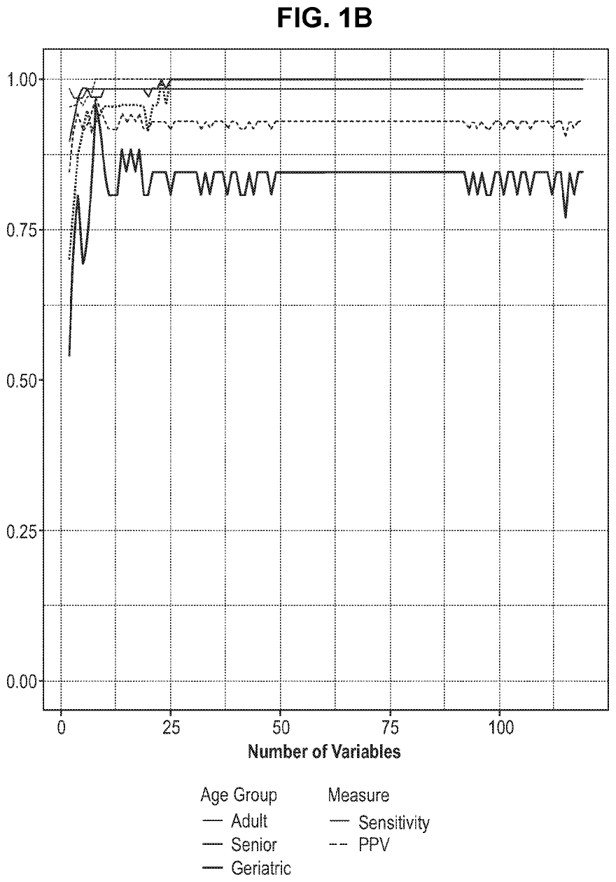

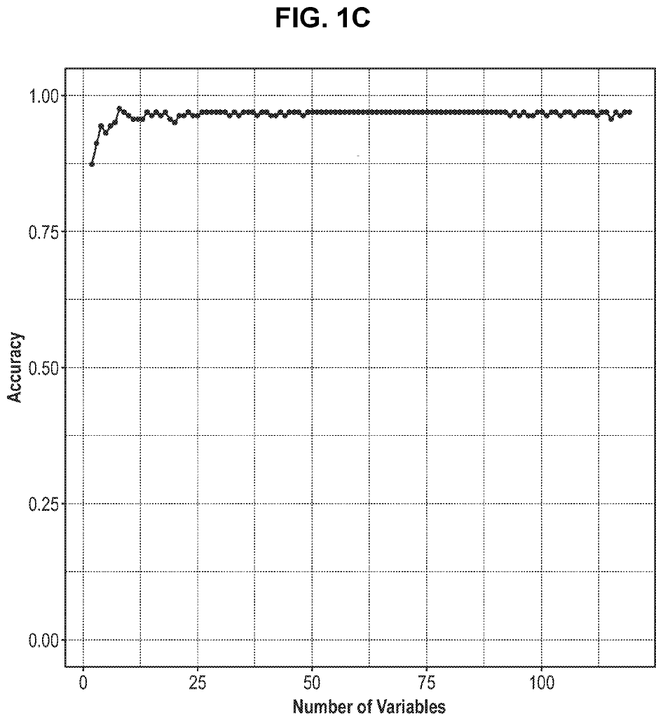

The bacterial gene sequences representative of OTUs and bacterial species were, differentially descriptive of life stage group or age of the cats (FIGS. 1A-1C) and thus contributed with greater or lesser accuracy to the assignment of the samples to the correct life stage group for the cat that had produced the sample. FIGS. 1A-1C shows the variation in the position of the individual OTUs in the prediction of life stage group based on the compositional characteristics of the faecal microbiome.

A combination of up to 30 OTUs or bacterial species were detected that enabled accurate assignment of the samples to life stage/age group of the cat (FIGS. 1B, 1C and FIG. 6 (Table 1.3)). The accuracy in assignment of sample to group peaked with 7 OTUs or species at 97.45%, with accuracies ranging between 94.9 and 96.8 thereafter with increasing numbers of species or OTUs added to the predictive model. Given the individual variation within the microbiome of cats, however these organisms are likely to have additional utility within the wider population and hence are included within the predictive model of life stage/age based on the compositional characteristics of the faecal microbiome.

### Method

The method involves the extraction of DNA from a freshly produced faecal sample from a feline by a means such as the QIAamp Power Faecal DNA kit (Qiagen) and subsequently the use of molecular biology techniques to assess the detection rate and abundance of the combination of the bacterial species described below (FIGS. 4 and 5 (Tables 1.1 and 1.2)). The combination and abundance of the organisms detected allow the assessment of the biological age status of the microbiome and assessment of whether the microbiome components observed in faeces of the animal are descriptive of an immature or older than expected microbiome relative to the actual age of the animal. After DNA extraction and sequencing of the DNA/RNA by techniques such as 16S rRNA/rDNA amplicon, shotgun, metagenome, Illumina, nanopore or other DNA/RNA sequencing techniques is conducted on genetic material extracted from faeces, the relative abundance of the sequences descriptive of the organisms in FIG. 4 (Table 1.1) or DNA sequences within 95% identical to those in FIG. 5 (Table 1.2) is made.

Briefly, sequence data obtained from the test sample is clustered into groups of sequences with 98%-100% identity and a reference sequence from the clusters which represent >0.001% of the total sequences is then used to either 1) assign taxonomy, gene function through database homologues or to determine the nature of the biomarker through homology searches of DNA databases such as the Greengenes or Silva or the NCBI non-redundant nucleotide sequence database for comparison to known DNA sequences for species held within the databases or 2) compared to the DNA sequences given in FIG. 5 (Table 1.2) below.

The number and abundance of the organisms, sequences or biomarkers identified from within the combinations described in FIG. 6 (Table 1.3) descriptive of the biological age status of the microbiome are then used to describe the sample in terms of the group with which they best fit and hence to assign a biological age status to the microbiome of the test cat.

### Example 2—A Further Method of Detecting the Biological Age Status of the Faecal Microbiome in Cats

Bacterial OTU (species) detection and relative abundance data for the 113 individual OTUs as well as the ‘rare’ group, was analysed by partial least squares discriminate analysis (PLS-DA) to assess likeness characteristics data within the samples taken from the cohort of adult, senior and geriatric cats. In the resulting correlation plot samples were clustered in a supervised manner directed by the likeness of samples based on detection of OTUs and their relative abundance.

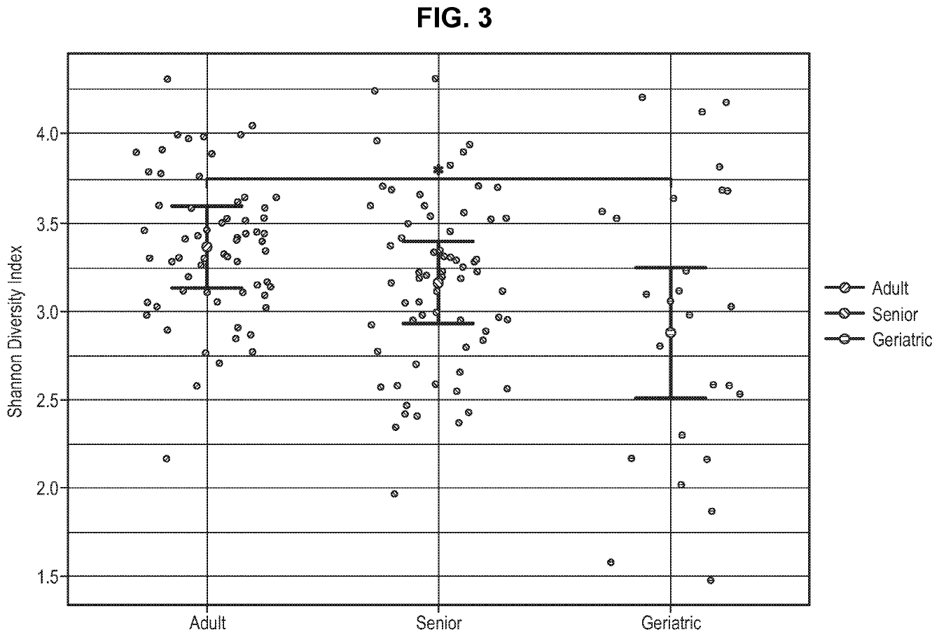

The multivariate supervised dimension reduction and regression method, detected correlations between the bacterial detection and abundance data that supported clustering of the samples almost exclusively by cohort group. Analysis plots demonstrated two geriatric cat samples from a single cat (Jazz) clustered more closely with senior cats while two adult samples from individual cats Jwoww and Pouncey fell within the otherwise geriatric cluster. Samples otherwise clustered according to life stage despite the unsupervised nature of the PLS clustering. Hence, by this method, similarly to the Random forest analysis, the bacterial gene sequences detected were observed to be differentially descriptive of life stage group or age of the cats (FIG. 3).

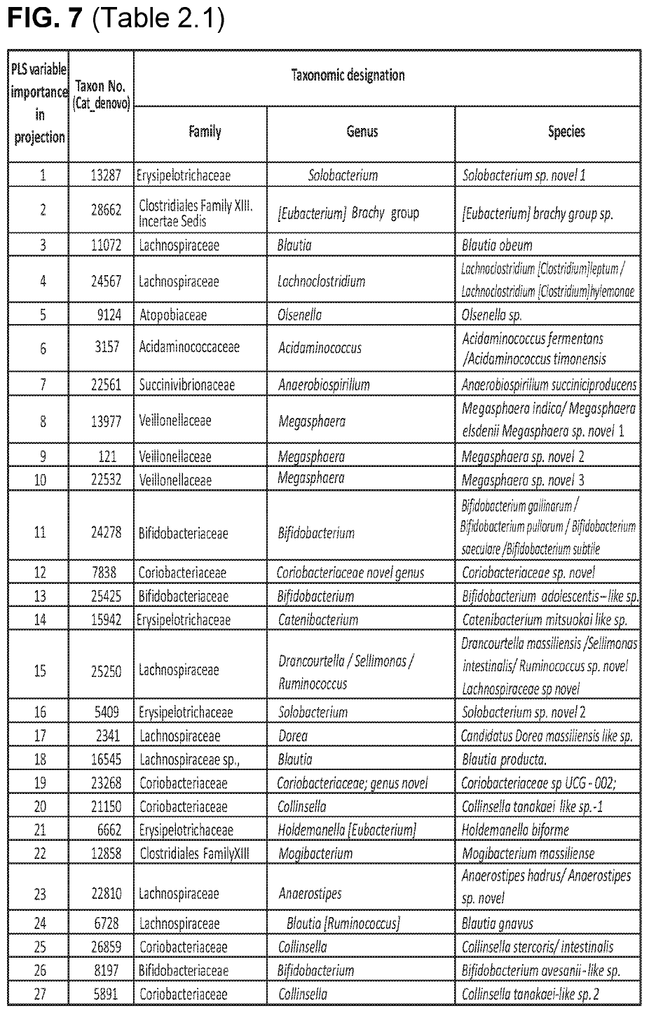

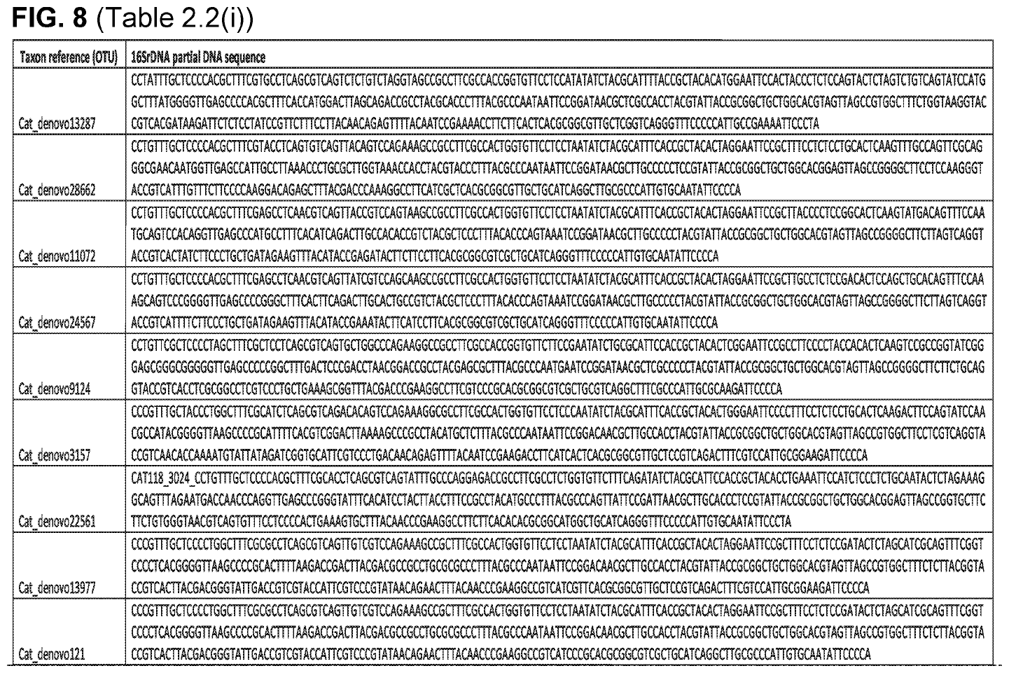

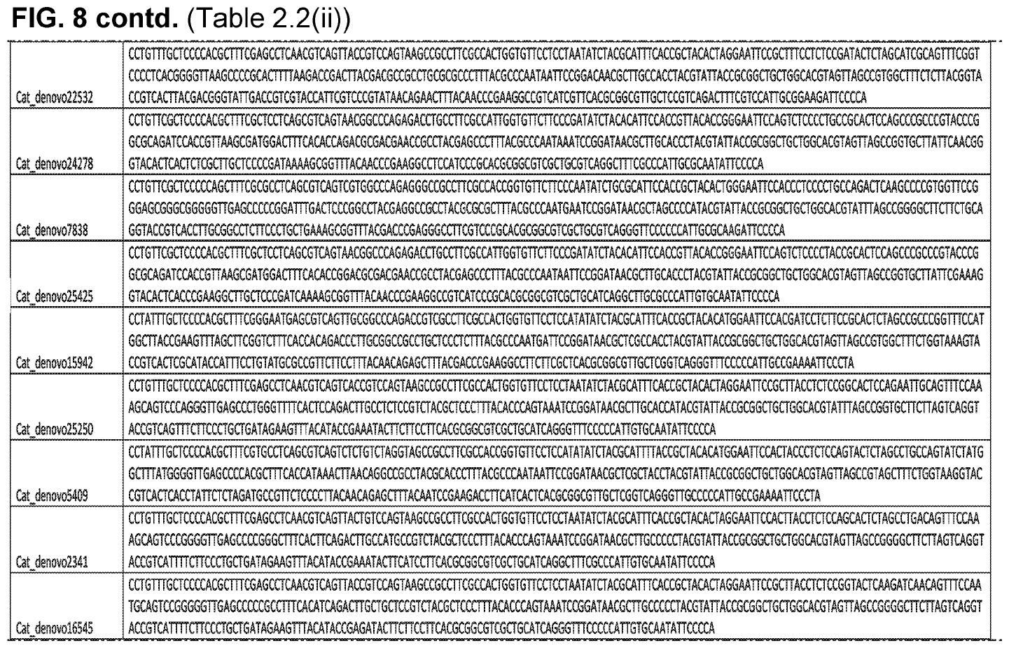

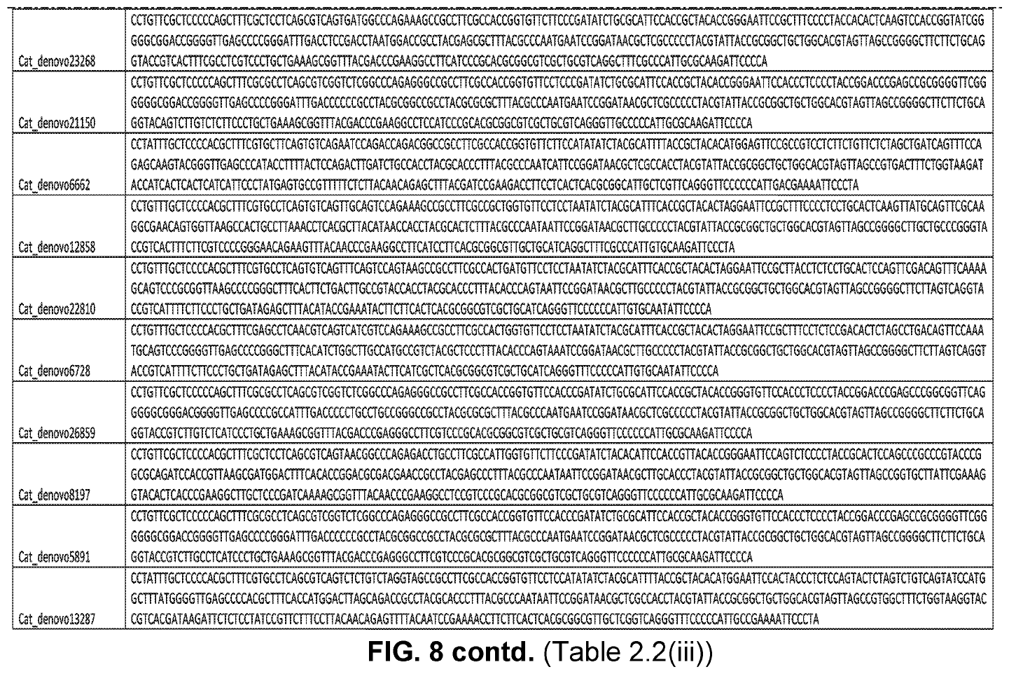

When the bacterial species most influential in driving separation of samples in the PLS-DA analysis (variable importance in projection; VIP score >1) were used in the analysis, this subset of 27 bacterial species or OTUs was similarly found to accurately cluster samples into life stage groups (FIG. 3). The analysis of the subset generated clusters with five senior samples and one adult sample inappropriately clustered into the geriatric group and two geriatric samples clustering with samples from senior cats. These 27 bacterial OTUs were therefore indicative of separation in the microbiota of the adult, senior and geriatric life stages (FIGS. 7 and 8 (Tables 2.1 and 2.2)).

### Method

A method to identify the age status of the faecal microbiome in cats is based on partial least squares discriminate analysis (PLS-DA) of the same microbial sequence and abundance data from the faeces of cats and comparison to control datasets from the faeces of cats in difference age or life stage groups. The method can be applied to the whole microbiome community abundance data or using a subset such as that shown in FIG. 7 (Table 2.1). Alternatively other methods for detection of the presence and abundance of the species highlighted in FIG. 7 (Table 2.1) and described by gene sequences in FIG. 8 (Table 2.2) (or >95% similar) could be assessed and similarly compared to control datasets from cats of different ages/life stages to determine the age status of the microbiome compared to the actual chronological age of the cat and thus to determine the consistency between actual age and microbiome age status.

In greater detail, the method involves the extraction of DNA from a freshly produced faecal sample by a means such as the QIAamp Power Faecal DNA kit (Qiagen) and subsequently sequencing of the DNA by techniques such as 16S rDNA amplicon, shotgun, metagenome, Illumina, nanopore or other DNA or RNA sequencing techniques. In the case of 16S rDNA analysis following DNA extraction from the faecal sample and Illumina sequencing of the V4-V6 region using DNA oligonucleotide primers (sequence (319F: CAAGCAGAAG ACGGCATACG AGATGTGACT GGAGTTCAGA CGTGTGCTCT TCCGATCT (SEQ ID NO: 43) and 806R: AATGATACGG CGACCACCGA GATCTACACT CTTTCCCTAC ACGACGCTCT TCCGATCT (SEQ ID NO: 44)) the resulting DNA sequences were clustered at 98% identity and abundant species (representing >0.001 of the total sequences) were then assessed for their relative proportions. Other regions descriptive of bacterial taxon can also be used.

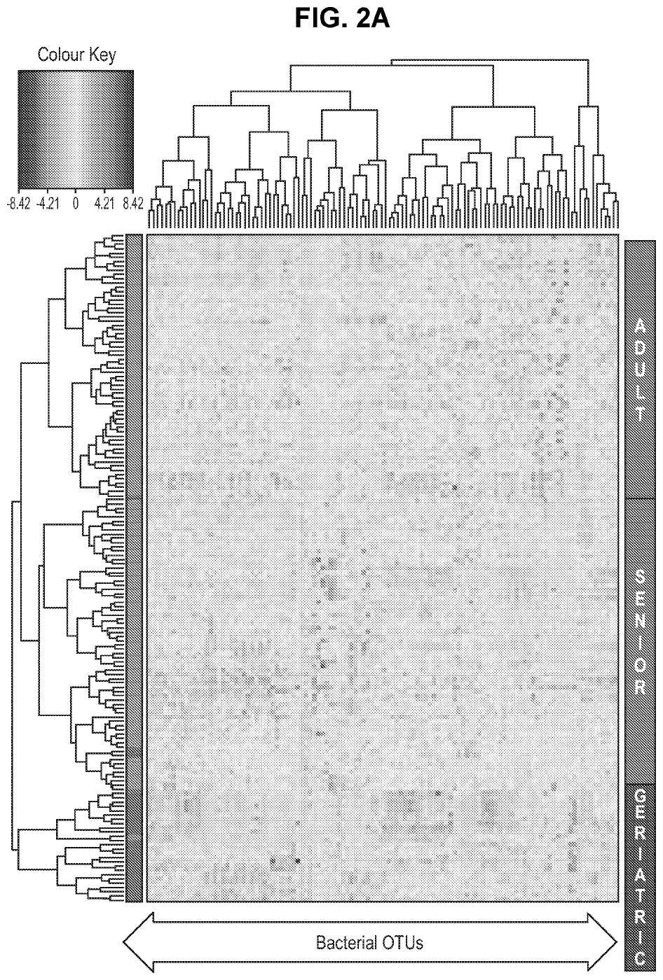

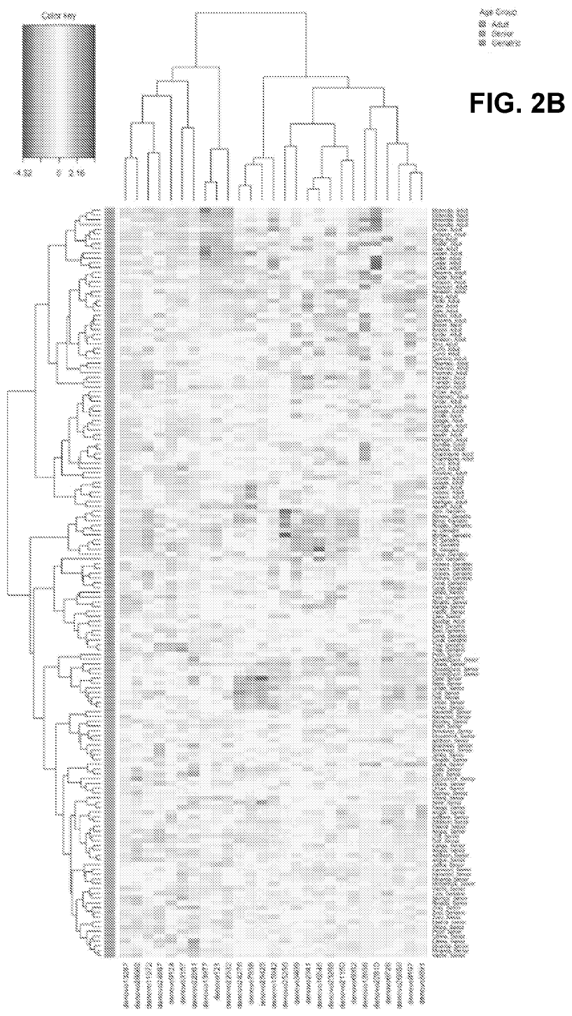

In any of the analysis methods resulting DNA/RNA sequences are clustered to species (>98% ID) level. Clusters, termed operational taxonomic units (OTUs) are then assigned a reference sequence (ie the sequence most similar to the rest of the cluster) and reference sequences are compared to either the sequences below in FIG. 8 (Table 2.2) or are assigned taxonomy based on database searches using the reference sequence to interrogate the Greengenes or Silva or the NCBI non-redundant nucleotide sequence databases. Those OTUs designated through database searches as the species described in FIG. 7 (Table 2.1) or with 16S rRNA gene sequences with 98% or greater similarity to those in FIG. 8 (Table 2.2) are analysed for detection of the relative abundance of the sequences descriptive of that OTU compared to the total sequences per sample. Alternatively any genetic sequences or biomarkers characteristic or descriptive of the organisms described in FIG. 7 (Table 2.1) can be used to determine relative abundance compared to the total microbiota community. The relative abundance data for the organisms described in FIG. 7 (Table 2.1) and FIG. 8 (Table 2.2) are then analysed by partial least squares discriminate analysis compared to the same bacterial content from control animals in kitten, adult, senior and geriatric life stages. Detection of the microbiome age status is made for the test sample by comparison to control samples based on which life stage group the sample (s) cluster with (e.g., See FIG. 2B). The life stage/age cluster the sample sits within describes the biological age status of the sample and this is then compared to the actual chronological age of the cat the sample has been produced by. The key elements are described in FIGS. 7 and 8 (Tables 2.1 and 2.2) and an example analysis supporting detection of biological age of the microbiome compared to control samples is given in FIG. 2B. This figure demonstrates separation of the feline microbiome with life stage suggesting that the composition of the microbiome is altered with age and hence the biological age status of the sample can be indicative of the health of the microbiome and the gastrointestinal resilience of the animal.

### Example 3—A Method of Detecting the Age Status of the Feline Gut Microbiome Based Bacterial Diversity in the Faecal Microbiome

Shannon diversity was calculated for each sample based on the OTU/taxon count and relative abundance according to the equation below. Shannon diversity was modelled using a linear mixed effects model with a fixed effect of age group and random intercept of pet. Pairwise comparisons of the life stage groups were performed with a controlled familywise error rate of 5%.

$$Shannon Index ( H ) = - {\sum }_{i = 1}^{s} {p}_{i} ln {p}_{i}$$

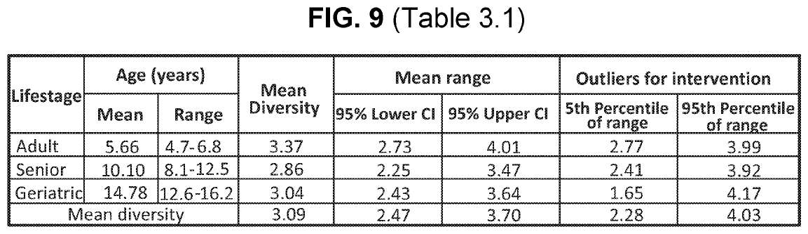

Assessment of Shannon diversity in the microbiota of cats from adult senior and geriatric cats yielded diversity estimates (FIG. 3 and FIG. 9 (Table 3.1)). A statistically significant difference was observed in the Shannon diversity of the faecal microbiome of adult cats (mean age 5.66 years) compared to those in the geriatric population (mean age 14.78 years).

### Method

The method involves the extraction of DNA from a freshly produced faecal sample by a means such as the QIAamp Power Faecal DNA kit (Qiagen) and subsequently the use of molecular biology techniques to detect the 16S rDNA or rRNA present or other genetic features enabling determination of bacterial abundance and taxon or species richness of the microbial community in faeces or other gastrointestinal sample. After DNA/RNA extraction from freshly produced faeces and genetic sequence analysis by techniques such as 16S rRNA/16S rDNA amplicon, shotgun, metagenome, Illumina, nanopore or other sequencing technique, the resulting sequences are clustered to species (>98% ID) level and the relative abundance of each species is determined for the individual OTUs as a proportion of the total. The total number of sequences or OTUs and OTU relative abundance data are then used to calculate diversity which accounts for both abundance and evenness of the species detected. Any diversity calculation can be used such as Shannon diversity or other alpha diversity calculations or alternatively beta diversity can be used. Shannon Diversity can be calculated by the following method:

$$Shannon Index ( H ) = - {\sum }_{i = 1}^{s} {p}_{i} ln {p}_{i}$$

After the determination of diversity of the microbiome for the test sample using functions such as Shannon diversity indices or other alpha or beta diversity assessment (including total OTU number with the sample) diversity can be compared to standardised samples from healthy control populations within the same life stage (see FIG. 9 (Table 3.1)) and to animals of similar age with chronic gastrointestinal enteropathy, IBD, acute or chronic diarrhoea or other gastrointestinal symptoms.

The interpretation of microbiome age status is based on the level of the diversity detected in the faeces of the cat in context of the animal's life stage compared to the control samples and can include previous analyses of the same cat at different ages from stored samples or previously collected data. In the case of assessment within the individual over time the gastrointestinal health of the cat can be monitored over time by testing/assessment of the gut microbiome periodically at intervals such as 6 monthly or annual or following particular events such as gastrointestinal upset, or travel. The results of assessment of the microbial diversity can then be compared with the previous results or cumulative (averaged) results from the previous assessments of the microbiome from the individual cat.

### REFERENCES

[1] Frank et al. (2007) Proc. Natl. Acad. Sci. USA 104, 13780-13785.
[2] Gevers et al. (2014) Cell Host Microbe 15, 382-392.
[3] Ni et al. (2017) Sci. Transl. Med. 9, eaah6888.
[4] Kostic et al. (2013) Cell Host Microbe 14, 207-215.
[5] Johnson and Foster (2018) Nature Reviews Microbiology, October; 16(10):647-655
[6] Kirchoff et al. (2018) PeerJ Preprints 6:e26990v1
[7] Sharon et al. (2013) Genome research, 23(1), pp. 111-120.
[8] Hart et al. (2015) PLoS One. Nov. 24; 10(11):e0143334
[9] WO2018/006080
[10] Gennaro (2000) Remington: The Science and Practice of Pharmacy. 20th edition, ISBN: 0683306472.
[11] Molecular Biology Techniques: An Intensive Laboratory Course, (Ream et al., eds., 1998, Academic Press).
[12] Methods In Enzymology (S. Colowick and N. Kaplan, eds., Academic Press, Inc.)
[13] Handbook of Experimental Immunology, Vols. I-IV (D. M. Weir and C. C. Blackwell, eds, 1986, Blackwell Scientific Publications)
[14] Sambrook et al. (2001) Molecular Cloning: A Laboratory Manual, 3rd edition (Cold Spring Harbor Laboratory Press).
[15] Handbook of Surface and Colloidal Chemistry (Birdi, K. S. ed., CRC Press, 1997)
[16] Ausubel et al. (eds) (2002) Short protocols in molecular biology, 5th edition (Current Protocols).
[17] PCR (Introduction to Biotechniques Series), 2nd ed. (Newton & Graham eds., 1997, Springer Verlag)
[18] Current Protocols in Molecular Biology (F. M. Ausubel et al., eds., 1987) Supplement 30
[19] Smith & Waterman (1981) Adv. Appl. Math. 2: 482-489.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A method of determining the microbiome age status of a feline, comprising
(a) detecting one or more bacterial species in a sample obtained from the feline;
(b) comparing the one or more bacterial species in the sample to a control data set; and
(c) determining the microbiome age status.

2. The method of claim 1, wherein the control data set comprises microbiome data of felines at different times or life stages.

3. The method of claim 2, wherein the control data comprises microbiome data from at least two, at least three, or four life stages of a feline selected from the list consisting of a kitten, a youth feline, an adult feline, a senior feline and a geriatric feline.

4. (canceled)

5. The method of claim 1, wherein the method comprises quantitating the one or more bacterial species.

6. The method of claim 1, wherein the bacterial species from genera selected from the group consisting of: Solobacterium, Olsenella, Anaerobiospirillum, Blautia, Holdemanella, Collinsella, Bifidobacterium, Eubacterium, Ruminococcus, Dialister, Bacteroides, Lactobacillus, Adlercreutzia, Subdoligranulum, Clostridium, Mogibacterium, Sellimonas, Streptococcus, Subdoligranulum and Lachnoclostridium, or bacterial species from the family Lachnospiraceae.

7. The method of claim 6, wherein the bacterial species are selected from the group consisting of Solobacterium sp., Olsenella sp. Marseille-P2300, Anaerobiospirillum succiniciproducens, Blautia gnavus, Holdemanella biforme, Collinsella bouchesdurhonensis, Bifidobacterium sp., Eubacterium brachy, Collinsella tanakaei, Collinsella stercoris, Blautia obeum, Eubacterium cylindroides, Dialister/Bacteroides xylanisolvens, Lactobacillus ruminis, Adlercreutzia sp., Subdoligranulum sp., Clostridium perfringens, Mogibacterium massiliense, Sellimonas sp., Streptococcus luteciae, Lachnospiraceae sp., Subdoligranulum variabile, Dialister succinatiphilus, Lachnoclostridium [Clostridium] leptum, Lachnoclostridium [Clostridium] hylemonae and Eubacterium coprostanoligenes.

8. The method of claim 7, wherein the bacterial species has a 16S rDNA with at least about 95% identity to the sequence of any one of SEQ ID NOs: 1-30.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The method of claim 6, further comprising detecting or quantitating Eubacterium sp., Collinsella sp. Blautia sp., Dialister/Bacteroides sp., Lactobacillus sp., Adlercreutzia sp., Subdoligranulum sp.; preferably detecting or quantitating Eubacterium brachy, Collinsella tanakaei, Collinsella stercoris, Blautia obeum, Eubacterium cylindroides, Dialister/Bacteroides xylanisolvens, Lactobacillus ruminis, Adlercreutzia sp. and Subdoligranulum sp.

23. The method of claim 22, wherein the bacterial species have a 16S rDNA with at least about 95% identity to the sequence of any one of SEQ ID NOs: 9-18.

24. The method of claim 1, wherein step (b) comprises correlating the one or more bacterial species in the sample to a control data set using partial least squares discriminate analysis.

25. The method of claim 24, wherein the bacterial species are from genera selected from the group consisting of Solobacterium, Eubacterium, Blautia, Lachnoclostridium, Olsenella, Acidaminococcus, Anaerobiospirillum, Megasphaera, Bifidobacterium, Coriobacteriaceae, Catenibacterium, Drancourtella, Sellimonas, Ruminococcus, Dorea, Collinsella, Holdemanella, Mogibacterium and Anaerostipes.

26. The method of claim 25, wherein the bacterial species are selected from the group consisting of Solobacterium sp., Eubacterium brachy, Blautia obeum, Lachnoclostridium [Clostridium] leptum, Lachnoclostridium [Clostridium] hylemonae, Olsenella sp., Acidaminococcus fermentans, Acidaminococcus timonensis, Anaerobiospirillum succiniciproducens, Megasphaera indica, Megasphaera elsdenii, Megasphaera sp. Bifidobacterium gallinarum, Bifidobacterium pullorum, Bifidobacterium saeculare, Bifidobacterium subtile, Coriobacteriaceae sp., Bifidobacterium sp., Catenibacterium mitsuokai, Drancourtella massiliensis, Sellimonas intestinalis, Ruminococcus sp, Lachnospiraceae sp., Dorea sp., Blautia producta, Coriobacteriaceae sp, Collinsella sp., Holdemanella biforme, Mogibacterium massiliense, Anaerostipes hadrus, Anaerostipes sp., Blautia gnavus, Collinsella stercoris/intestinalis, Bifidobacterium sp. and Collinsella tanakaei.

27. The method of claim 26, wherein the bacterial species has a 16S rDNA with at least about 95% identity to the sequence of any one of SEQ ID NOs: 1, 8, 11, 29, 2, 31, 3, 32, 33, 34, 35, 36, 37, 38, 21, 28, 39, 40, 41, 9, 5, 20, 42, 4, 10, 7 and 15.

28. (canceled)

29. (canceled)

30. (canceled)

31. (canceled)

32. (canceled)

33. (canceled)

34. (canceled)

35. (canceled)

36. (canceled)

37. A method of monitoring a feline, comprising a step of determining the microbiome age status of the animal on at least two time points by
(a) detecting one or more bacterial species in a sample obtained from the feline;
(b) comparing the one or more bacterial species in the sample to a control data set; and
(c) determining the microbiome age status.

38. The method of claim 37, wherein the two time points are at least 6 months apart.

39. (canceled)

40. (canceled)

41. (canceled)

42. (canceled)

43. A method of assessing the microbiome age status of a feline to determine whether an intervention is required, comprising
(a) quantitating two or more bacterial species in a sample obtained from the feline;
(b) determining the relative abundance of said bacterial species;
(c) comparing the relative abundance determined in step (b) to that of a control data set; wherein if the comparing of step (c) indicates a difference in microbiome age status to actual age of the feline, an intervention is recommended.

44. (canceled)

45. The method of claim 43, wherein the control data set comprises bacterial species diversity data from a plurality of other felines of the same life stage as the feline; or bacterial species diversity data from the same feline at one or more different time points, preferably separated by at least 6 months.

46. (canceled)

47. The method of claim 43, wherein step (c) comprises calculating the Shannon index diversity of the bacterial species by the formula:
$$Shannon Index ( H ) = - {\sum }_{i = 1}^{s} {p}_{i} ln {p}_{i}$$

48. (canceled)

49. (canceled)

50. The method of claim 43, wherein the intervention is a dietary change, a supplement, a functional food, a nutraceutical or a pharmaceutical composition.

51. (canceled)

52. (canceled)

53. The method of claim 1, wherein the sample is a faecal sample, or a sample from the gastrointestinal tract.

54. (canceled)

55. (canceled)
